(19) United States
(12) Patent Application Publication
(10) Pub. No.: US 2021/0195888 A1
(43) Pub. Date: Jul. 1, 2021

(54) PROCESS OF CRYOPRESERVATION OF STEM CELLS OBTAINED FROM THE PULP OF DECIDUOUS TEETH

(71) Applicants: Jose Ricardo Muniz Ferreira, Vitoria City (BR); Brunella Sily de Assis Bumachar, Vila Velha City (BR); Alexandre Jose Serafim, Vitoria City (BR)

(72) Inventor: Jose Ricardo Muniz Ferreira, Vitoria (BR)

(73) Assignees: Jose Ricardo Muniz Ferreira, Vitoria City (US); Brunella Sily de Assis Bumachar, Vila Velha City (BR); Alexandre Jose Serafim, Vitoria City (BR)

(21) Appl. No.: 16/071,162

(22) PCT Filed: Sep. 11, 2017
(86) PCT No.: PCT/BR2017/000110

§ 371 Date: Jul. 19, 2018

(30) Foreign Application Priority Data
Oct. 3, 2016 (BR) 102016023038-1

(51) Int. Cl. A01N 1/02; C12N 5/0775

(52) U.S. Cl. CPC A01N 1/0221; C12N 5/0664

(57) ABSTRACT

PROCESS OF CRYOPRESERVATION OF STEM CELLS OBTAINED FROM THE PULP OF DECIDUOUS TEETH is a process of collection of stem cells from deciduous teeth for freezing and storing, comprises the sequence of procedures from the collection of the deciduous teeth to the storage of stem cells in favourable conditions to its application when and if necessary, and for the operationalization of the process a Collection Kit was developed, in order to allow the Dentist to perform the collection in its own practice according to pre-set technical protocols and timing for sending, which was called the R-Crio Collection Kit; finally the opening of a further possibility of protocol from collecting until the storage and subsequent thawing of the stem cells form the deciduous pulp for application in therapies and studies involving Regenerative Biomedicine.

The present invention refers to the process of cryopreservation of stem cells obtained from the pulp of deciduous teeth and subsequent thawing in conditions suitable for autologous/allogeneic use in therapeutic attempts and in research, more precisely to a process of treatment that encompasses an appointment prior to extraction of the tooth, selection, collection, preparation of solutions, isolation, expansion, quality control, quarantine, cryopreservation and thawing of mesenchymal stem cells obtained from the pulp of deciduous teeth. Through this process it will be possible to open a new possibility of protocol foreseeing from the collection to the storage and subsequent thawing of Stem Cells from the deciduous pulp for application in therapies and studies involving Regenerative Medicine.

During the research and development phase of the process, numerous studies were conducted in several databases, being identified PI 0513773 A, under the title of “Stem Cells obtained from the pulp of deciduous or permanent teeth and the tooth germ able to produce human bone tissue”. This technology encompasses the isolation, growth, application in the “in vitro” production of autologous bone with the preservation of this living bone and its application in the reconstruction of bone tissue in patients.

The process of cryopreservation of stem cells obtained from the pulp of deciduous teeth regards a sequence of procedures in suitable conditions, in such a way that the expanded cells could be used in therapeutic initiatives when and if necessary. For the operationalization of this process, it was developed a collection kit that allows the dentist to perform the collection in their own clinic, in accordance with pre-set technical protocols and shipping timelines, which is called R-Crio Collection Kit.

The process of cryopreservation of stem cells obtained from the pulp of deciduous teeth may be better understood through the detailed description in line with the following accompanying Figures, wherein:

FIG. 01 Presents an illustration of the tooth and the sectional area for the process of cryopreservation of stem cells obtained from the pulp of deciduous teeth.

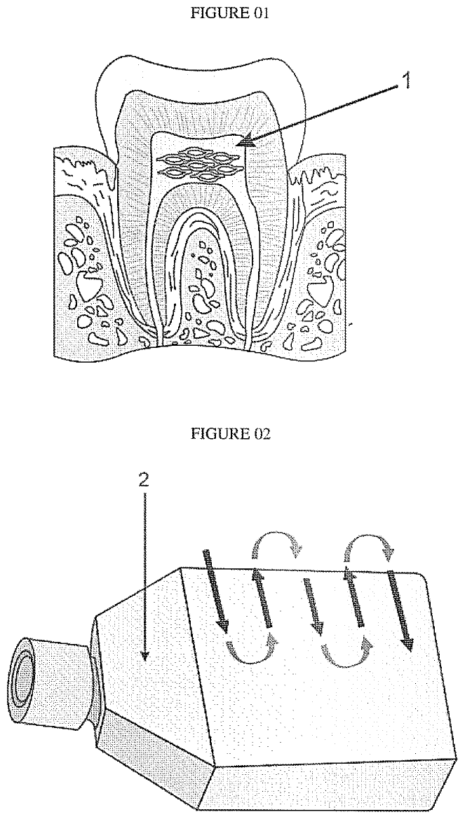

FIG. 02 Presents an illustration of the Shake Flask for the process of cryopreservation of stem cells obtained from the pulp of deciduous teeth.

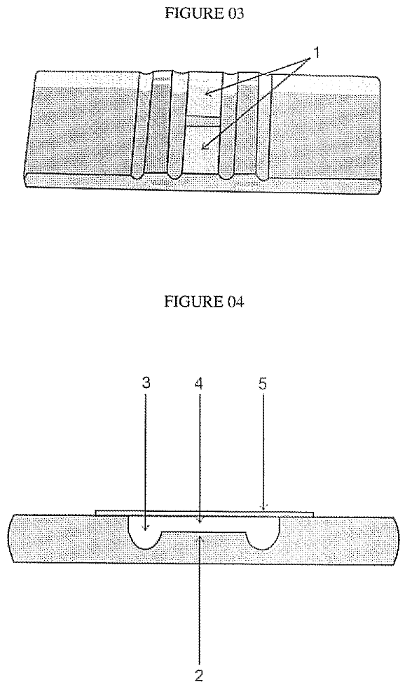

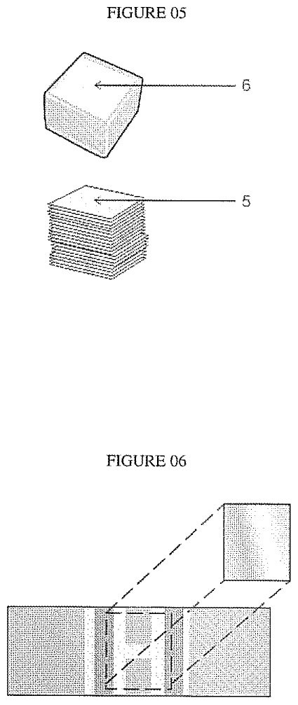

FIG. 03 Presents an overview indicating the Neubauer Chamber—Sample Insertion Chambers (A).

FIG. 04 Presents an overview of the Neubauer Chamber.

FIG. 05 Presents one of the coverslip boxes and coverslips.

FIG. 06 Shows the coverslip coupling on the sample insertion chambers.

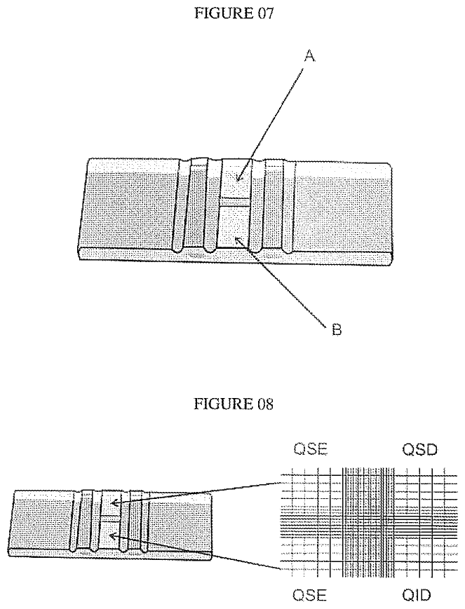

FIG. 07 Shows the insertions (A or B) of the Neubauer counting chamber.

FIG. 08 Presents an illustration of the Neubauer chamber squares.

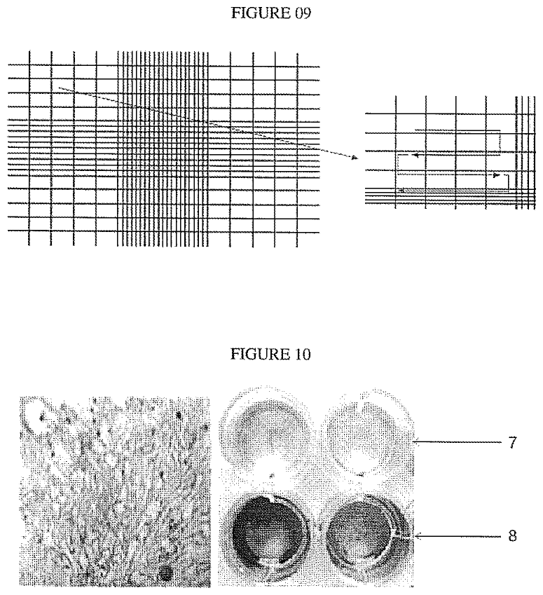

FIG. 09 Presents an illustration of the Counting and Cell Viability of Neubauer Chamber squares.

FIG. 10 Presents an analysis provided by the Color of Calcium deposits and Macroscopic Vision of the plate, indicating negative (7) and positive (8) controls.

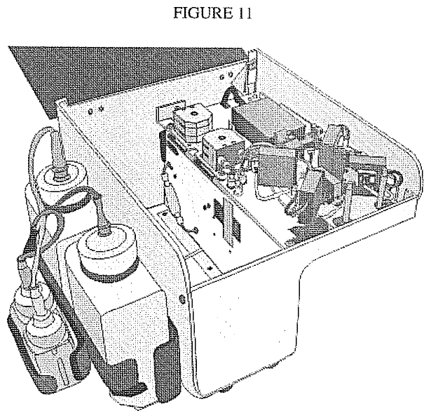

FIG. 11 Presents an overview of the equipment used in the process of cryopreservation of stem cells obtained from the pulp of deciduous teeth.

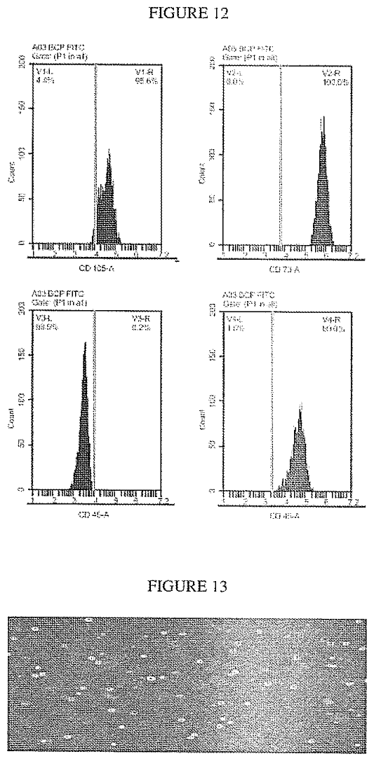

FIG. 12 Presents an example of the generated histograms and their results as a percentage.

FIG. 13 Presents an illustration of the non-adherent punctuate cells.

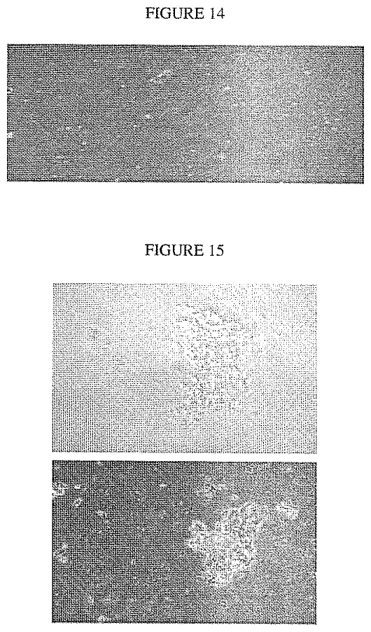

FIG. 14 Presents an illustration of some points with adherent non scattered cells.

FIG. 15 Presents an illustration of adherent or semi-adherent CFUs.

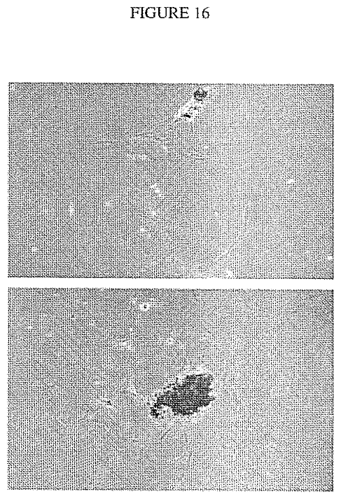

FIG. 16 Presents an illustration of adherent CFUs releasing scattered cells.

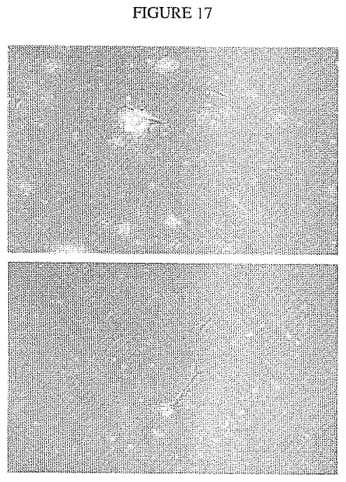

FIG. 17 Presents an illustration of rare points of scattered cells.

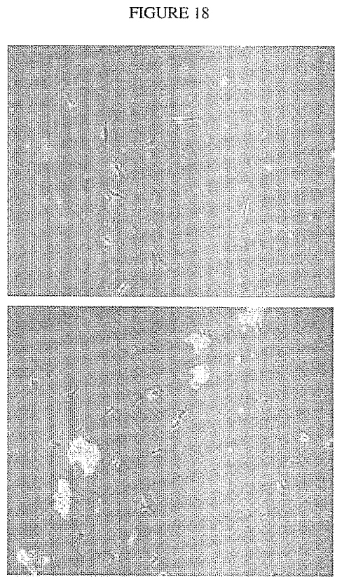

FIG. 18 Presents an illustration of scattered, fusiform, sparse cells.

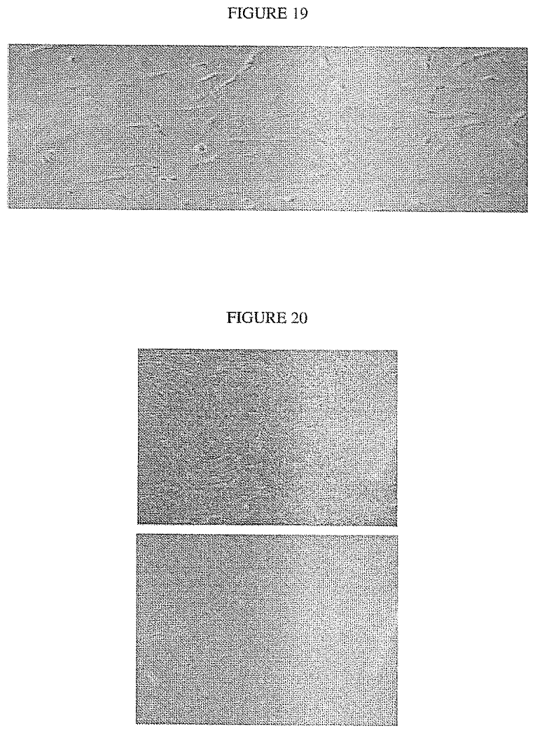

FIG. 19 Presents an illustration of several points with scattered cells growth.

FIG. 20 Presents an illustration of some points with high scattered cells growth.

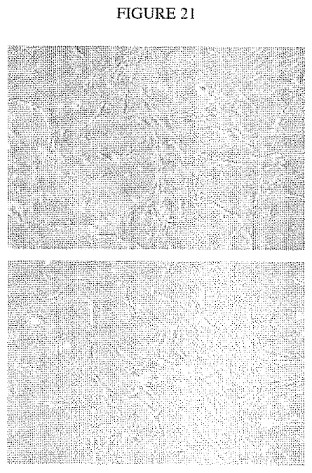

FIG. 21 Presents an illustration of low confluence.

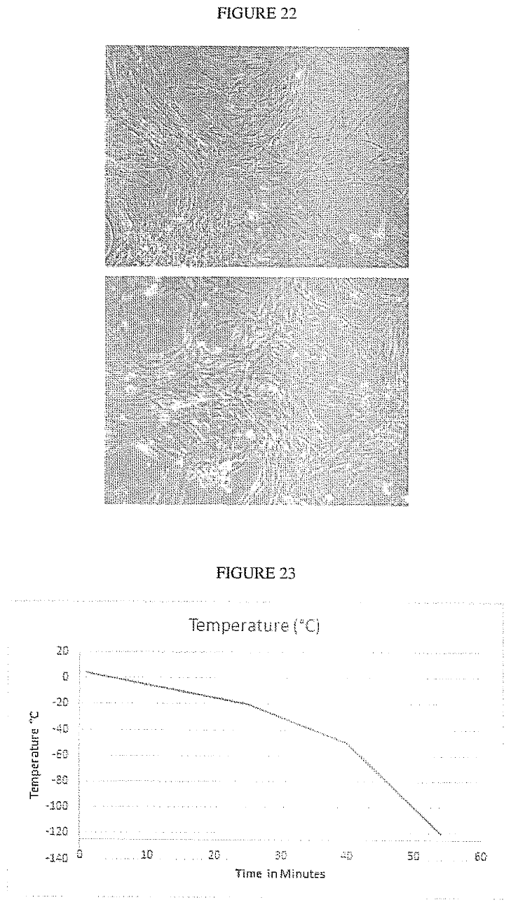

FIG. 22 Presents an illustration of a cell fit to passage or cryopreservation.

Detailed description of the procedures of the process of collection for freezing and storage of Stem Cells (2) from the pulp of deciduous teeth and of the composition of the R-Crio Collection Kit.

The entire process follows the sequence described below.

Appointment Prior to Tooth Extraction:
The appointment should be performed by a dentist accredited with a Cellular Technology Center licensed by the competent regulatory bodies.
In the appointment, the dentist must perform the Anamnesis (serological exam), indicating which tooth could be extracted and what is the most suitable timing, taking into account that the tooth must have at least a third of the root and be healthy, free of active infectious diseases.
These requirements are necessary to avoid the extraction of deciduous teeth with contaminated stem cells or not feasible for the expansion process.
The dentist must perform a periapical radiography of the tooth to be extracted and request the serological exam as follows:
The tooth extraction should be performed by a dental surgeon accredited with a Cellular Technology Center licensed by the competent regulatory bodies.

Selection:
The dental surgeon must select for extraction a deciduous dental element with at least ⅓ of reminiscent root. That way, a larger pulp volume is expected, and the pulp will not have had direct contact with the oral cavity and will not be contaminated by the present microbiota. That conduct aims to decrease the risk of contamination of the pulp content. Besides that, the stem cells isolated in this phase are expected to have larger potential and quality to be multiplied (expanded) and differentiated.
It must be verified, in a radiographic exam, the presence of the permanent dental element in infra-bone position corresponding to the space occupied by the deciduous dental element in process of exfoliation. In case of agenesis of the corresponding permanent element, the extraction of another deciduous dental element should be considered, so the correct occupation of the spaces created by the loss of deciduous teeth by the permanent teeth is not compromised.
The deciduous dental element must be free from any kind of active infection, to guarantee the conformity and larger predictability of the processes of isolation, expansion and quality control of the present mesenchymal stem cells.
Children between 6 and 12 years old, as a general rule, are in a period of mixed dentition and because of that should be assisted by a dentist for the indication of the deciduous tooth to be extracted in suitable conditions in accordance with the proposed protocol.
At the moment of tooth extraction, the child must be free of local diseases of acute systemic diseases.
Positive results for the presence of infectious diseases are not considered to be impediments to the performance of the whole process of isolation, expansion, quality control and cryopreservation of the stem cells. An external protection for the cryotube (double protection) is required as a safety measure in these cases.

Serological Exam Analysis

Aims to establish the parameters for analysis of the requested serological exam of the beneficiaries.

### Definitions and Abbreviations

AG HBS: Hepatitis B virus surface antigen.
Anti-HBc IgM: Antibodies IgM against hepatitis B virus core antigen.
Anti-HBc: Total antibodies against hepatitis B virus core antigen.
Anti-HBS: Antibodies against hepatitis B virus surface antigen.
Anti-HCV: Antibodies against hepatitis C virus.

### CMV:Cytomegalovirus.

FTA-ABS IgM: Absorption of FTA IgM (Fluorescent Treponemal Antibody), Immunofluorescence for syphilis.
FTA-ABS: Absorption of FTA (Fluorescent Treponemal Antibody), Immunofluorescence for syphilis.
HBS AG: Hepatitis B virus surface antigen.
HBV: Hepatitis B virus.
HCV qualitative RNA: Research of the Hepatitis C virus RNA.
Qualitative HIV: Quantification of the viral RNA for HIV-1.
HTLV I-II: Human T cells lymphotropic virus.

### IgG: Immunoglobulin G.

### IgM: Immunoglobulin M.

HCV: Hepatitis C virus.
T. cruzi IgM: Antibodies IgM anti-Trypanosoma cruzi.
T. cruzi IgG: Antibodies anti-Trypanosoma cruzi.

Exam Checking

The results are in conformity with the following criteria:

| Infectious diseases | Exam name | Expected result |
|---|---|---|
| Syphilis | VDRL | Nonreactive |
| | FTA-ABS | Nonreactive |
| | FTA-ABS IgM | Nonreactive |
| Chagas disease | T. cruzi IgG | Negative |
| | T. cruzi IgM | Negative |
| Hepatitis B (HBV) | AG HBs ou HBs Ag | Nonreactive |
| | Anti-HBc total | Nonreactive |
| | Anti-HBc IgM | Nonreactive |
| | Anti-HBc IgG | Nonreactive |
| | Anti-HBs | Reactive or Nonreactive |
| Hepatitis C | Anti-HCV | Nonreactive |
| | HCV- qualitative RNA | Not detected |
| HIV 1 e 2 | Anti-HIV 1-2 (antigen/antibody) | Nonreactive |
| | qualitative HIV (RNA) | Nonreactive |
| | HIV IgG/IgM | Nonreactive |
| HTLV I/II | HTLV I-II | Nonreactive |
| Cytomegalovirus (CMV) | Cytomegalovirus IgG | Reactive or Nonreactive |
| | Cytomegalovirus IgM | Nonreactive |

Shipping the Sample Packaging Box

First, the thermal box should be sanitized with a disposable cloth embedded in alcohol 70% to eliminate any contaminating agent. 5 units of reusable ice will be used: 2 units of 20×9 cm and 3 units of 22×16 cm. The ice should be kept in a freezer until the time of preparation of the box. Next, two Ice Foams of 20×9 cm will be put on the bottom of the thermal box. Wrap the other two ices of 22×16 cm on the sides of the box.

Place the tube rack. Place absorbent material, for possible spillage, between the primary and secondary packaging. Then place an ice of 22×16 cm. Before placing the tubes in the rack, verify if they are whole, do not present leak, and are identified with lot and validity. Place, inside de box, some units of secondary packaging (ziplock) and 2 plastic seals for the thermal box, which will be used in return. When the samples storage is finished, close the lid and seal the thermal box on the metal handle.

Collection

The collection of deciduous teeth is done by accredited dentists, able to perform the procedure of extraction of the deciduous tooth in accordance with the protocols set by the cellular technology center. It is necessary that the child parents or legal guardian and the responsible clinical sign the service contract. It is recommended that, prior to extraction, the child be healthy and free of oral infectious diseases. It is also recommended and desirable that, before extraction, the tooth be inspected to identify possible cavities or infections. Immediately following the extraction, the tooth should be immersed in chlorhexidine gluconate solution 0.12% for 1 minute. After this period, the tooth is transferred to the tube with transport medium and stored in a refrigerator until the time of shipping or, in case the tooth be already shipped to destiny, inserted in the box of the R-Crio Collection Kit.

Health Questionnaire

Shipping

The collection kit containing the extracted tooth packed into the tube with the culture medium and antibiotics, along with a copy of the service contract, should be sent to the laboratory (cellular technology center) within 48 hours of tooth collection.

Sample Transport

### Definitions and Abbreviations

Licensed Service Centers: Clinic with qualified professional for deciduous teeth extraction.
Primary packaging: packaging that is in direct contact with the biological material to be transported, composed of container, involucre or any other form of protection, removable or not, which is intended for packaging, holding, containing, covering or wrapping the biological material to be transported, also called internal packaging.
Category A: infectious biological material whose exposure to it may cause permanent disability of fatal illness, endangering human or other animal life signalized as UN 2814 or UN 2900 if it endangers only animals.
Category B: infectious biological material not falling within category A, classified as “biological substance of Category B” UN 3373, being inserted in this group samples from patients suspected of known to contain infectious agents causing diseases in humans.
Exempt Human Specimen: includes biological materials from healthy individuals who have undergone professional judgment based on clinical history, individual symptoms and characteristics, as well as local endemic conditions that ensure the minimum likelihood of the biological material containing pathogenic microorganisms, even if these materials have not previously been submitted to tests for markers of diseases transmissible by blood, following the guidelines of the World Health Organization (WHO).
Secondary packaging: intermediate packaging, placed between the primary packaging and the tertiary packaging, for the purpose of containing the primary packaging.
Tertiary packaging: external packaging, used exclusively for the protection of cargo in handling operations (loading, unloading and transport) and storage.
Shipping company: company that provides transport services competent for the transport of biological material.

Procedure

Classification of the Biological Risk of Biological Samples:

The deciduous tooth is a biological material with a minimum likelihood of containing infectious agents that could compromise transportation. The dental surgeon is trained to evaluate signs and symptoms, clinical history, situations of risk exposure in order to ensure through the anamnesis the quality of the tooth that will be transported.

Thus, according to WHO, Exempt Human Specimen is exempt from regulations on hazardous items, however, as they are biological, the risk will not be zero. In this way care is taken with this biological material during transportation.

Contracts with Shipping Companies

The transport of biological material will only be carried out by a carrier that meets the IATA. UN and WHO standards for biological transport (IATA/48th Edition. NU/13th Edition. WHO/13th Edition) or RDC n. 20 of 2014 (Brazil), proven by presentation of standard operating procedure.

Transportation of Thermal Boxes without Samples to the Licensed Service Centers.

The following materials shall be present in the thermal box:
Primary packaging: 20 ml tube in polypropylene or polyethylene with conical bottom containing culture medium.
Secondary packaging: Plastic bag with sealing.
Tertiary packaging: Thermal boxes for transportation of biological material, with safety lock.
Absorbent material for possible leakage.
Self-adhesive label for sample identification.
Recyclable ice in rigid and flexible packaging.
Plastic rack for test tubes.

Transportation of Biological Samples to the Laboratory

Air and Ground Transportation:

The material must reach the R-Crio Laboratory in a maximum of 48 hours and transportation must take place according to the regulations in force in each country for biological samples.

Sample Receipt

### Definitions and Abbreviations

Biological sample: part of the biological material of human origin used for laboratory analysis.
Rejected sample: biological sample out of specification.
Thermal box: box for carrying biological sample (portable refrigerator type) made of polyethylene or similar.
ID—Sample Identification, Number generated by system or patient initials.
Foreign bodies: Particles suspended in the medium.

Procedure

Receipt of Tooth Sample:

The tooth sample arrives at the laboratory always in polypropylene thermic box with identification of the laboratory, equipped with thermometer calibrated to verify the temperature of transport.

Open the thermal box carefully to maintain the integrity of the samples. Any sample arriving at R-Crio has a secondary packaging with absorbent material that prevents liquid spreading if the tube is not properly closed. This pack is discarded in garbage identified as Glove/Mask, for further incineration.

Verify that the samples has the following conditions:

### 1. Insulated Thermal Box Sealing

2. Secondary packaging in the sample (Dispose in appropriate place—Glove/Mask)

### 3. Identification by ID.

4. Tube integrity with loss of significant amount of medium.
5. No leakage.
6. Absence of foreign bodies.
7. Clear culture medium without cloudiness.
8. Tooth integrity.

Criteria for Exclusion of Tooth Sample:

One of the above conditions, if not met, immediately impedes processing of the tooth sample: Lack of Identification of the tube by ID. The other conditions do not prevent the processing of the sample, but will be identified as non-conforming to the described process that guarantees the quality of the sample and will only be approved for cryopreservation if all stages of the isolation, expansion and quality control processes are fulfilled with satisfactory results.

Printing of Labels for all Material Used in the Process

Generate the labels for the following materials and refer them to the sanitation:
Culture Bottle
Incubator Map
Digestion Tube
Waste Disposal
Reserve.

Filling in the Biological Sample Receipt Form

After all the conferences, complete the Biological Sample Receipt Form which will be filled with the documentation generated at the end of processing and send it along with the sample to the processing industry.

Biological Sample Receipt Form:

Organization of Deciduous Teeth Samples

Samples should be organized in ascending order of numbering of the work orders, which represents the extraction sequence of the teeth, thus respecting the processing schedule, without exceeding the stipulated maximum limit of 48 hours of sample storage. Store the samples under refrigeration at a maximum of 10° C. until transferred to the Sanitation sector.

Preparing Solutions and Mediums

Its purpose is to standardize solutions in the laboratory for use throughout the process.

Procedure

All solutions are prepared as described below, according to the masses described in the specific form and labelled. Manipulated solutions are valid for one month after preparation and the aliquots have the validity provided in the package, provided that the storage conditions are maintained. There are some solutions of Medium that have specific validity, but that is described in its specific form. Below is the basic preparation of some solutions:

Preparation of Sterile Liquids:
In a biological safety booth, place all the solutions that will be used, previously sanitized with alcohol 70°GL.
With the aid of serological pipettes and micropipettes, transfer aseptically the appropriate sterile container solutions.
Register on specific form and label it.
Ex: Complete Xeno-free Medium and Solutions A and B.

Note: The tips used in the process are sterile and should preferably have a filter.

Preparation of Sterile Solution with Powdered Raw Material:
Weigh the sample in an analytical balance with the aid of a spatula.
Dispense the heavy mass into a volumetric flask or suitable container.
Homogenize until dissolved.
Transfer the volume to volumetric glass and complete the volume.
If the final volume of the solution is low, adjust it with a volumetric pipette.
In a biological safety cabinet, filter the solution in a 0.22 μm membrane filter, dispensing it in a suitable sterile vial.
Register on specific form and label it.
Ex: Collagenase and Thiosulfate powder.

Sterilization of Non-Sterile Liquids:
In a biological safety cabinet, filter the solution in a 0.22 μm membrane filter, dispensing it in a suitable sterile vial.
Register on specific form and label it.
Ex: Ready solutions like Sodium Thiosulfate and Chlorhexidine.

Fractioning and Caution of Components of Solutions and Cultural Media

Before preparing each solution or culture medium, the components are fractionated to facilitate the preparation and guarantee the quality of the solution, avoiding cycles of thawing of the components. Fractionation is carried out in a Biological Safety Cabinet with sterile materials and using aseptic techniques. The tubes are identified with specific label with the name, lot and the validity of the solution. The fractionation is recorded in a specific form. Below are the procedures and care in the fractionation and storage of the solutions. Other volumes may be prepared provided they maintain the proper proportion.

Fractionation of Supplement for Xeno-Free Medium

This solution may have a cloudy appearance after thawing. It should be used immediately after thawing and thaw cycles should be avoided. Storage is carried out from light.
Defrost the solution in a refrigerator (2 to 8° C.) overnight.

In a biological safety cabinet, perform the fractionation as described:
Separate adequate quantity of sterile tubes.
Using a micropipette coupled with a sterile 1000 μL filter tip, transfer 0.5 ml aliquots of stock solution into sterile tubes.
Identify the tubes with the name of the solution, lot and validity.
Cover them with foil.
Condition in a proper rack.
Register the fractionation in the Solution Fraction Record.
Store the solution in a range of −20° C. to −5° C.

Human AB Serum Fractionation

This solution has no available data on toxicological information and has been tested for infectious agents. It comes from donors from the United States of America. It has a yellowish-brown coloration.
Thaw the solution at room temperature.

In a biological safety cabinet, perform the fractionation as described:
Separate sterile tubes with 2.0 ml volume and sterile serological pipettes or sterile tips.
Using a micropipette coupled with a sterile 1000 μL filter tip, transfer aliquots of 1.25 ml of the stock solution into sterile tubes.
Identify the tubes with solution name, lot and validity.
Condition in a proper rack.
Record the fractionation in the Solution Fraction Record.
Store the solution at −20° C.

Fractionation of L-Glutamine 200 MM

L-Glutamine is an amino acid extremely sensitive to thawing cycles, acidic and basic pH's and high temperatures, and it can lose up to 25% of its activity if such variations occur. Therefore, fractionation and freezing of the solution are extremely important. When thawing this solution, it may appear white. The solution has a clear appearance when heated to 37° C. The storage temperature of the stock solution is −20° C. After opened, it should be kept refrigerated and maintained at 4° C. for a maximum of two weeks.
Thaw the solution at room temperature of if necessary in a water bath at 37° C.

In biological safety cabinet, perform the fractionation as described:
Separate an appropriate amount of sterile Eppendorf tubes.
Using a micropipette coupled with a sterile 1000 μL filter tip, transfer 0.51 aliquots of the stock solution into sterile tubes.
Identify the tubes with solution name, lot and validity.
Condition in a proper rack.
Record the fractionation in the following Solution Fraction Record.
Store the solution at −20° C.

Fractionation of Penicillin/Streptomycin

The stabilized antibiotic solution has 10.000 Units of Penicillin and 10 mg/ml of Streptomycin. It is widely used for addition in culture medium for cell growth. The solution is clear and translucent.
Thaw the solution at room temperature.

In a biological safety cabinet, perform the fractionation as described:
Separate an appropriate amount of sterile Eppendorf tubes.
Using a micropipette coupled with a sterile 1000 μL filter tip, transfer 0.6 ml aliquots of the stock solution into sterile tubes.
Identify the tubes with solution name, lot and validity.
Condition in a proper rack.
Record the fractionation in the following Solution Fraction Record.
Store the solution at −20° C.

Solution Fraction Record 2:

Fractionation of Amphotericin

Amphotericin solution is an antifungal and its appearance is yellowish and clear. It is not uncommon to form precipitates in aqueous solutions, but the solution remains suitable for use. In cell culture, at 37° C. it is stable for 3 days. In refrigerator with a temperature between 2 to 8° C., it is stable for approximately 2 to 3 weeks. For long periods keep frozen at −20° C.
Thaw the solution at room temperature.

In a biological safety cabinet, perform the fractionation as described:
Separate an appropriate amount of sterile Eppendorf tubes.
Using a micropipette coupled with a sterile 200 μL filter tip, transfer 0.6 ml aliquots of the stock solution into sterile tubes.
Identify the tubes with solution name, lot and expiration date.
Condition in a proper rack.
Register the fractionation in the Solution Fraction Record 1.
Store the solution at −20° C.

Preparation and Specific Caution of Solutions and Culture Media

Some solutions or culture media have specific care and particularities in the preparation, fractionation, expiration date and storage. Details on the preparation of these solutions are given below.

Complete Xeno-Free Medium Preparation

The xeno-free medium is composed of 5 solutions, being: Human AB Serum: Xeno-free supplement; L-Glutamine: Penicillin/Streptomycin and Xeno-free Basal Medium. This medium will be used in the expansion and cryopreservation of cells, the latter being added by Dimethyl Sulfoxide.
Defrost the components of the medium, that is, the fractionated aliquots.
Perform the preparation as described in the item “Preparation of Sterile Liquids” and also according to specific instructions on the solution record form 2.
Fraction 50 ml of the medium per tube.
Label it.
This medium is valid for two weeks after preparation and should be stored in a refrigerator with a temperature of 2 to 8° C., protected from light.

Preparation of 1×PBS

The PBS solution is composed of Sodium Chloride (NaCl), Potassium Chloride (KCl), Dibasic Sodium Phosphate (Na2HPO4) and Monobasic Potassium Phosphate (KH2PO4). It is used to prepare the solutions A and B used in the sample washing step.
Carry out the preparation as described in the item “Preparation of sterile solution with powdered raw material” and also according to specific instructions on the record form.

Record Form 3:
Weight the product and transfer to Becker or suitable bottle.
Dissolve the reagents with 80% of the final volume of the solution.
Correct pH to 7.4 with Sodium Hydroxide of Hydrochloric Acid.
Complete the volume of the solution with ultra-purified water.
Filter solution through a 0.22 μm membrane.
Fraction 50 mL of the solution per tube per tube.
Label it.
This solution is valid for 01 month after preparation and should be stored in a refrigerator with a temperature of 2 to 8° C.

Preparation of Solution A

Solution A is composed of 1×PBS pH 7.4, added with Penicillin/Streptomycin and Amphotericin. It is used in the first washing step of the sample. The validity of this solution is dependent on Amphotericin, which after thawing is stable for a maximum of three weeks.
Defrost the components of the medium, that is, the fractionated aliquots.
Perform the preparation as described in the item “Preparation of Sterile Liquids” and also according to specific instructions on the record form 4.
Fraction 5 mL of the medium per tube.
Label it.
This solution expires after three 03 weeks and should be stored in a refrigerator with a temperature of 2 to 8° C.

Record Form 4

Preparation of 0.2% Chlorhexidine Solution

The 0.2% Chlorhexidine solution is usually purchased in its liquid form and in the working concentration. It is used in the second washing step of the sample. The solution should be stored away from light.
Carry out the preparation as described in the “Sterilization of Non-Sterile Liquids” and also according to specific instructions on the record form 5.

Record Form 5:
Fraction 5 mL of the medium per tube.
Cover the tubes with foil.
Label it.
The stock solution and aliquots have their validity according to the manufacturer's instructions provided that their storage is as described on the label.
Store the solution at room temperature away from light, heat and moisture.

Preparation of 0.1% Sodium Thiosulfate

The Thiosulfate solution is used in the third step of the sample washing process. It can be made in two ways, depending on the physical property of the stock bottle, i.e. liquid or solid stock solution. The minimum bacterial activity is achieved when the pH of this solution is between 9 and 10, so 0.1 g of Sodium Carbonate is added to each liter of solution, ensuring the correct pH.

The preparation of the solutions for both cases is given below. Considerations: 1 mole of pentahydrate Thiosulfate contains 248.18 g/L, but we must discount the PM of the water, as follows:
5 H2O=18×5=90 g, therefore:
248.18−90=158.18 g of Thiosulfate per liter, i.e., a 0.1M solution contains approximately 1.6% Sodium Thiosulfate, whereby the Concentration and Dilution formula can be used.
A solution of 0.1% Sodium Thiosulfate is equivalent to a 0.01M mole solution, so the Molarity formula can be used to prepare the solution if it is solid.

Preparation of Sodium Thiosulfate. Liquid Stock 0.1 mol/L or 0.1N:
Perform the preparation as described in the item “Preparation of Sterile Liquids” and also according to specific instructions in the record form 6.

Record Form 6:
Complete the volume of the solution with ultra-purified water.
Filter solution through 0.22 μm membrane.
Fraction 5 mL of the medium per tube.
Label it.
Cover with foil.
This solution expires after 1 month and should be stored at room temperature in the range of 15 to 25° C.

Preparation of Sodium Thiosulfate, solid stock: Considerations: The solid form of Sodium Thiosulfate is very hygroscopic, that is, it has great ease in adsorbing water. Therefore, do not leave the bottle open for long periods. Open for the time required to weigh the sample.
Perform the preparation as described in the item “Preparation of Sterile Solution” with powdered raw material and also according to specific instructions on the record form 7.

Record Form 7:
Weight the product rapidly and transfer to a suitable volumetric flask or vial.
Complete the volume of the solution with ultra-purified water.
Filter solution through a 0.22 μm membrane.
Fraction 5 mL of the medium per tube.
Label it.
Cover it with foil.
The solution expires after 01 month and should be stored at room temperature in the range of 15 to 25° C.

Preparation of Solution B

Solution B is composed of 1×PBS pH 7.4, added with Penicillin/Streptomycin. It is used in the last washing step of the sample.
Defrost the components of the medium, that is, the fractionated aliquots.
Perform the preparation as described in the item “Preparation of Sterile Liquids” and also according to specific instructions in record form 8.

Record Form 8:
Fraction 5 mL of the medium per tube.
Label it.
This solution expires after 01 month and should be stored in a refrigerator with a temperature of 2 to 8° C.

Collagenase Preparation

The collagenase solution is used for the digestion of the pulp after extraction, allowing the cells to be removed for cultivation. Its activation is carried out by the Calcium atom at a concentration of 5 mM per mole of enzyme. It suffers loss of its activity through thaw cycles. It is used after the extraction of the pulp. In some cases, there may be a need to increase the concentration of this solution, which may range from 0.3 to 0.5%.
Perform the preparation as described in the item “Preparation of Sterile Solution” with powdered raw material and also according to specific instructions on the record form 9.

Record Form 9:
Weight the product and transfer to a suitable volumetric flask or vial.
Add 80% of the final volume of the HBSS solution without Calcium and without Magnesium or 1×PBS.
Homogenize until the product is dissolved.
Fill the volume with the same solution.
Filter solution through a 0.22 μm membrane.
Fraction 1.5 mL of the medium per tube.
Label it.
This solution expires after 1 month and should be stored in a freezer at −20° C.

Preparation of General Solutions

This form is used for the preparation of solutions not covered by this procedure (record form 10). The reagents used are registered in the specific fields of the form. If a preparation methodology other than those covered in this procedure is used, describe it in the field of observations. If necessary, attach the solution label or some pertinent information on the back of the page.

Record Form 10:

### Solution

Examples of solutions using this form:
Basal Medium.
PBS solution from flask with ready raw material.
Sodium hydroxide.
Hydrochloric acid.

Calculations and formulas:
Below are the formulas and examples of calculations to aid in the preparation of solutions:

Gram Equivalent Formula:

Gram Equivalent (GE) of a chemical element is the ratio between atom-gram (A) or mole by its valence (v), number of ionisable hydrogens or hydroxyls in the compound under consideration.

Formula:GE=A/v
Where: A (mole₁) is the atomic mass of the atom or of the sum of the elements, obtained in the periodic table.
V is the valence, or number of electrons in the last layer.

### EXAMPLES

Calculation of gram equivalent for Na+ and Ba++: Na−GE=23/1=23 g: Ba−GE=137/2=68.5 g.

Calculation of gram equivalent for sulfuric acid—H2SO4 and for hypophosphorous acid—H3PO2: H2SO4−GE=98/2=49 g: H3PO2−GE=66/1=66 g.

Calculation of gram equivalent for sodium thiosulfate pentahydrate: Na2S2O3−GE=248.18/1=248.18 g.

Note: Before preparing a sodium thiosulfate solution, it is necessary to subtract the water mass from the total molar mass:

Calculation: Na2S2O3−5H2O=248.18−90=158.18 g

That is, the molar mass of the actual sodium thiosulfate is 158.18 g, that is 63.73%.

Normality Formula:

Normality (N) or normal concentration is the ratio of the number of gram equivalents of the solute to the volume of the solution, in liters.

N=m/GE·V(L)
Where: m=mass of the solute in grams;
GE=Gram Equivalent;
V=Volume in Liters.

### Examples

Calculation of Normality for sodium thiosulfate: N=248.18/248.18.1=1N

How many grams of sodium thiosulfate is in a solution with normality N=0.1?
0.1N=m/248.18 0.1=24.8B of sodium thiosulfate per liter of the solution.

Molarity Formula:

Molarity or molar concentration is the ratio of the number of moles of the solute to the volume of the solution, in liters. M=[m/MWV]*(g)/(L)
Where: m=mass of the solution in grams;
MW=mole mass or molecular weight;
V=Volume in liters.

### Examples

To prepare 500 ml of a 3M solution of NaOH, how many grams of the solute are needed? 3M=x/40*0.5 (L)=60 g of NaOH.

Concentration and dilution formula:C₁·V₁=C₂·V₂
Where: C₁=initial solution concentration;
V₁=initial solution volume;
C₂=final solution concentration;
V₂=final solution volume.

### EXAMPLES

What is the volume required to prepare 100 mL of a 0.2% chlorhexidine solution from a stock solution of 2%? 2%. X=0.2%. 100=10 mL.

General Observations:
All solutions are prepared according to this procedure and recorded in a specific form.
If the solution is photosensitive, cover it with foil.
When a solution is not contemplated in this procedure, use the General Solutions record, and if the preparation has any particularity, register in the field observations.
Delete the blank fields with a risk or the abbreviation N.A. (Not Applicable).
The labels of the original solution, when they are fractionated, are placed at the back of the page, if necessary.
When it is not possible to label the solution, make the lot and validity marking with indelible marker or specific label containing the solution name, lot and validity.

Solution Label Template:

Sample Processing

Sample processing is performed within 48 hours after sample extraction, thereby maintaining cell viability.

### Definitions and Abbreviations

PBS: Phosphate-buffered saline;
PBS P/S: Phosphate-buffered saline containing Penicillin/Streptomycin;
PBS P/S/A: Phosphate-buffered saline containing Penicillin/Streptomycin/Amphotericin;
UV: ultraviolet;
Xeno-free: Free of xenobiotics;
Basal Medium: Basic Culture Medium, ex: RPMI, DMEM, DPSCBM, etc;
RPMI medium: culture medium developed by Roswell Park Memorial Institute;
DMEM: Dulbecco's Modified Eagle's Medium;
DMSO: Dimethyl sulfoxide;
RT: Room temperature,
WB: Water Bath;
RPM: Revolutions per minute;
ATM: atmosphere

Procedure

Preparation for Sample Processing:

Inanimate Materials:
02 10 mL sterile graduated pipettes;
06 3.5 mL sterile Pasteur pipettes;
02 Packages of sterile wiper;
01 sterile 90×15 petri dish;
01 50 ml sterile falcon tube;
01 25 cm² culture bottle;
01 Pipette (semi-automatic and/or manual);
01 sterile cutting pliers;
02 sterile forceps,
01 thick tip pliers;
01 Gray curette;
01 Lima Kerr No. 25 or pulp excise rasp;
01 sterile scalpel

Solutions:
5 mL of solution A (1×PBS, Penicillin, Streptomycin and Amphotericin)
5 mL 2% chlorhexidine solution;
5 mL of 0.1% sodium thiosulfate solution
5 mL Solution B (1×PBS, Penicillin, Streptomycin)
2 mL of Xeno-free medium preheated to 37° C.;
3 mL of Basal medium preheated to 37° C.
2 mL of Solution B (1×PBS, Penicillin, Streptomycin)
3 mL of IX PBS solution
1 Vial 1.5 ml of 0.3% collagenase solution

Observations: Only the Inanimate Materials are placed inside the biosafety cabinet and exposed to U.V light.

Equipment Verification

All equipment to be part of the process shall be checked prior to sample processing for correct operation, cleaning and general maintenance. Below are listed the equipment that will be part of the process.
Biological safety cabinet;
Water bath;
Centrifuge;
CO2 incubator;
Semi-automatic pipette;
Stopwatch.

Processing of the Sample (Deciduous Tooth or Third Molar)

The sample processing is divided into 4 parts: Cleaning; Sectioning; Collection and Digestion of the Pulp and Culture. Except for the sample centrifugation steps, the entire procedure is performed in a biological safety cabinet.

The instruments to be used in the processing are packed in surgical paper and sterilized according to the relevant procedure. When receiving the kit, consisting of: 01 sterile cutting pliers; 02 sterile forceps, 01 thick tip pliers; 01 Gray curette; 01 Lima Kerr No. 25 or pulp excise rasp; and 01 sterile scalpel, check the integrity of the kit as well as the validity of the sterilization and record in the sample processing record form below.

Sample Processing Record Form:

Separate a shelf and place the tubes (Processing Kit) as follows.

### 1. Solution A;

### 2. Chlorhexidine 0.2%;

3. Sodium thiosulfate 0.1%;
4. Solution B (5 mL);
5. Solution B (2 mL), being identified with the label generated on receipt as Digestion Tube;
6. Collagenase 0.3%, thaw before use;
7. Basal medium, put in water bath at 37° C.;
8. 1×PBS, put in water bath at 37° C.;
9. Xeno-free medium, put in water bath at 37° C.

Before starting the process, identify the tube mentioned above (Solution B—2 mL), the 25 cm² culture bottle and the drop/cut tube (50 mL falcon tube, identified as disposal).

The procedure to be performed is detailed below.

Tooth Cleaning

The first step in the process is the cleaning of the tooth. To that end, four solutions are used:
Solution A (1×PBS, penicillin/streptomycin and amphotericin)
0.2% Chlorhexidine Gluconate;
0.1% v/v Sodium Thiosulfate;
Solution B (1×PBS, penicillin/streptomycin)

Perform tooth cleaning as described below:
Separate the tubes, in the sequence described above, with cleaning solutions on an appropriate shelf.
With the aid of sterile forceps remove tooth from the transport tube and transfer it to Solution A (penicillin/streptomycin/amphotericin).
Gently homogenize the tube for 1 minute.
After this period, remove the tooth with the forceps and gently remove excess solution.
Transfer the tooth to another tube containing the 0.2% chlorhexidine solution.
Gently mix the tube for 1 minute.
Remove the tooth again, remove excess solution and transfer to the next tube with the 0.1% sodium thiosulfate solution.
Gently mix the tube for 1 minute, remove the tooth and again remove the excess solution.
Transfer to PBS solution B (Penicillin/Streptomycin).
Homogenize again for 1 minute and transfer the tooth to sterile petri dish.

Sectioning

The second step is carried out cross-sectioning the tooth in the region between the crown and the root (cementoenamel junction) for access to the pulp chamber. Below is an illustrative picture of tooth structure, location of the sectioning area (1), location of the pulp chamber, according to FIG. 01.
Place a 50 ml sterile Falcon tube on an appropriate shelf and leave the lid half-open.
With the aid of sterile surgical straight forceps or suitable pliers, hold the tooth with one hand.
With the aid of sterile cutting pliers, place it at the cementoenamel junction and hold the tooth.
Remove the tube lid and insert the tooth, held by the cutting pliers inside the tube.
Section the tooth.

Pulp Collection and Digestion

The third step is the pulp collection and digestion. This step is carried out gently and with specific materials, thus ensuring the quality of the collected material.
Label a 15 mL conical bottom tube (unique beneficiary ID) containing 2 ml of solution B (PBS/Penicillin/Streptomycin).
With the aid of a rasp and/or curette (Lima Kerr 25 or 35 mm), remove all possible pulp and transfer it to a petri dish. Carry out the longitudinal cutting of the pulp with the aid of a sterile scalpel. Transfer it directly to the conical bottom tube containing 2 mL of solution B (PBS/Penicillin/streptomycin).
Homogenize the tube gently for a few seconds.
Centrifuge the tube for 5 minutes at 1000 RPM or 178G.
Add 1.5 mL of 0.3% collagenase enzyme sterile solution.
Proceed to incubation in water bath at 37±2° C. for 15 to 30 minutes. Visually inspect the bleaching and disintegration of the material inside the tube, characteristic of the pulp digestion.
At the end of the incubation, add 3 mL of basal medium (RPMI or DMEM) preheated to 37° C. in a water bath.
Homogenize the tube and centrifuge for 5 minutes at 1000 RPM or 178G.
Discard the supernatant.
Add 3 mL of PBS solution 1× and centrifuge the tube to for 5 minutes at 1000 RPM or 178G to wash the material.
Discard the supernatant.
The pellet will be used for the cultivation stage.

Cell Culture

The fourth step is the cell culture. In this step, material is transferred into a culture bottle and then sample incubation occurs under conditions suitable for expansion. Proceed as follows:
Identify a 25 cm bottle with the unique identification label of the beneficiary.
Resuspend tube contents with 1.5 to 2 mL of Xeno-free medium.
   This volume depends on the size of the material collected, where the volume of the medium is proportional to the volume/mass of pulp collected.
Homogenize the tube by suctioning and dispensing the tube volume and transfer all the contents to the 25 cm² bottle, gently dispensing it directly onto the bottle floor.
Homogenize the bottle so that the volume of the suspension spreads throughout its floor. To do this, perform movements as an “8” on the floor of the cabinet.
This bottle is to be incubated in an oven with a temperature of 37±1° C. and an atmosphere of 5% CO2.
If the bottle does not have a filter in its lid, leave it semi-open by slightly unlatching it when positioning it on the incubator shelf. Check the space before opening the incubator door.
Leave the bottle incubated for 3 to 7 days and after this period, proceed with monitoring and media exchange.

Monitoring and Exchange of Media

Aim: standardize samples' media monitoring and change during cellular growth phase and cell expansion, describing and detailing the steps conducted.

### Definitions and Abbreviations

Confluence—Decrease of the spaces between cells, with the formation of a layer of cells due to multiplication and increase in the number of cells in a culture flask.
CFU (Colony Forming Units).
Debris—cellular waste product.

Procedure

Preparing for the Procedure:

Inanimate Materials:
Suction hoses kit (Aspiramax);
0.2 ml sterile pipette tip without filter;
02 10 ml sterile serological pipets;
08 6 ml sterile Pasteur pipette;
01 50 ml conical bottom tube for disposal of residues;
02 Packages of Wipers.

Solutions:
Aliquot of Complete Xeno-free Medium preheated to 37° C.;
Aliquot of Solution B (1×PBS, Penicillin, Streptomycin) preheated to 37° C.;
Alcohol 70° GL.

Observations: Only inanimate materials are placed inside the cabinet and exposed to UV light. Solutions are wrapped in plastic bags and incubated at 37° C. in water bath.

Characteristics of Cells Observed During Monitoring

These characteristics are extremely important during cell growth and isolation monitoring, since they point out possible deviations in the process or prove the success of the process. Below are the main features:
Non-adherent punctate cells: refractive, rounded cells, i.e., whose microscope image resembles a point of light, but are not adhered to the bottle bottom and remain suspended in the bottle supernatant. FIG. 13.
Some spots with cells adhered, not scattered: round cells, refractive, in the initial process of adhesion, that is, they are still attached to the bottom of the bottle, but do not show fusiform and scattered formats. FIG. 14.
Adhered and/or semi-adhered CFU's: “pockets” or sets of round, refractive cells that are in the beginning of the process of releasing of scattered cells. FIG. 15.
Adhered CFU's releasing scattered cells: “pockets” or sets of round cells, fully adhered, refractive, surrounded by fusiform and scattered cells. FIG. 16.
Rare spots of scattered cells: adhered cells, scattered and fusiform in a few spots of the bottle. They are usually found before the start of their multiplication or after expansion passage. FIG. 17.
Scattered, fusiform cells, sparse: cells with the characteristics mentioned above in various spots of the bottle, but without confluence, that is, with large spaces between cells growth. They are usually found at the beginning of their multiplication. FIG. 18.
Several spots with scattered cell growth: cells with the characteristics mentioned above in various spots of the bottle and with beginning of confluence. FIG. 19.
Some points with large growth of scattered cells: cells with the mentioned characteristics, in distant points of the bottle, without the beginning of confluence due to the distance between the colonies. FIG. 20.
Low Confluence: cells at advanced stage of growth and multiplication with little space between cells and approximate confluency of 30 to 40%. They are still not apt for passage. FIG. 21.
Suitable for passage: cells with homogeneous growth in the whole bottle; at this stage, large quantities of cells form a “carpet” or “monolayer” of cells that cover the bottle bottom surface in a percentage between 60 and 90%. When cells are found in this stage of development passage to a larger bottle or cryopreservation should be performed. FIG. 22.

Schedule and Care During Monitoring

The schedule and care set forth below are extremely important to obtaining viable cells as well as to ensure the quality and effectiveness of cell growth.
Begin monitoring within 3 to 5 days after sample processing.
Clean the bottle with a cloth soaked in alcohol after removing the bottle from the incubator and before returning it to the incubator again.
Sample monitoring should be performed as soon as possible in order to not disturb and compromise the quality of the cells.
Do not exceed the time of 5 minutes when the sample is being observed under the microscope.
Perform the transportation of the bottle as gently as possible.
After 5 minutes, return the bottle to incubator and leave it there for 10 minutes before analyzing it again.
If the activity to be performed is not done after the observations, return the sample to the incubator.
After handling the sample and returning it to incubator, if the bottle does not have a ventilation system filter, check whether the bottle cap is unscrewed.
If fusiform cell adhesion is not verified within a 24-day incubation period, record the occurrence on the Non-adherent cells communication form and inform the person responsible for the area about the occurrence.

Non-Adherent Cells Communication Form:
If any deviation in the process, such as contamination, lack of growth or any deviation that precludes the process is verified, fill in the Sample rejection record form and notify the responsible of the area for relevant measures to be taken.

Sample Rejection Record Form:

Cell Growth Monitoring: (FIG. 02)
Check on the map of the incubator where the sample to be verified is.
Open the incubator, close the lid of the culture bottle and take the sample, in a gentle manner.
Transport the bottle gently in the horizontal position, taking care that the supernatant does not touch the lid.
Clean the base of the bottle with cloth soaked in alcohol 10° GL.
Fit the bottle on the microscope table and select the OX lens.
Start the stopwatch, preset to 5 minutes.
Adjust the focus and perform the observations.
Analyze the bottle as a whole to record a homogenous look of the bottle.
Follow the Technique for Homogenous Verification of the Bottle of Culture in Microscope described in FIG. 2, where the arrows represent the locations that must be observed.

Attention: Do not exceed the time of 5 minutes for the microscopic examination, in order not to disturb the evolution and the cellular growth. Long exposures to light can disrupt cell growth, as well as time away from the appropriate incubator can lower CO2 concentration and alter pH.

Physical and Electronic Records

Records are made on specific forms during and after the remarks, as described below:
Records of cell tracking observations are performed on the History and Next Steps form (Sample Rejection Record above). In this form, fill in the observations of the day, the activity that was performed and what will be the activity and expected date, for the next follow-up.

Photographic Records

The photographic records are made to characterize the stem cells and when it is necessary to follow the evolution of their growth, the confluence before the passage and the detachment of the cells during the passage or cellular expansion. This procedure can be modified according to the software of the equipment used, as well as the operating system. Regardless of this, records should be made for the assurance and traceability of the information. Below is the photographic registration procedure:
With the bottle positioned on the microscope table, select the desired lens.
Capture the image through the equipment software.
Create a folder with the patient ID.
Within this folder, if it is the first image to be registered, create a folder with the title Tracking, for example: 001-1 and, inside this one, a folder written “Monitoring”.
Within the Monitoring folder create a folder identified with the number of days the sample is incubated (i.e., 5 Days).
In this folder, identify the photo in the File Name field as the example: Beneficiary ID—Incubation Days—Sequential number (According to the number of photos captured), i.e., 333-15 days 1—First captured photo of this beneficiary in the Day 5.
In the Type field, select the JPEG or relevant format and click Save.

Medium Change

Medium changes begin with the beginning of sample monitoring, i.e., within 3 and 5 days of sample processing or if growth and cell adhesion are verified. There are two methods for performing medium change, one being the Medium Change with Suction Equipment and the other being Medium Change with Pipettes. Regarding the protocol used, there are also two types, one being the exchange of ⅔ of the medium and the other the Total Medium Volume change.

Below are the descriptions of medium change methods that can be used.

Medium Change with Suction Equipment

The suction hose kit is packaged in two parts, one containing only the hose and the other containing the hose, the quick coupling and the tip connection. The kit is supplied to the processing sector in appropriate and sterile package.
Insert the packaging with the hose, the quick coupler and the tip connection in a biological safety cabinet.
Open the other package in the external area, insert one end hose in the suction equipment and the other end in the biological safety cabinet.
Open the other package, containing the silicone hose, the suction tip and the quick coupling and connect the hose attached to the equipment.
Inside the cabinet, turn on the suction.
With the aid of the suction tip, connect the 200 μL sterile pipette tip.
Open the bottle and insert the suction tip deep enough to remove the medium from the bottle.
Proceed with the protocols described in the items Change of ⅔ of the medium and Total Medium Volume change.

Medium Change with Pipette:
Open the bottle, turn it so that the medium is on the opposite side of the growth and insert the pipette to the bottom of the bottle and remove the medium.
Disposal the medium appropriately.
Proceed with the protocols described in the items of Change of ⅔ of the medium or Total Medium Volume change.

Change of ⅔ of the Medium

This change is usually performed in the first 20 days of incubation, to accelerate the growth of cells due to paracrine factors released by them. The decision to use or not use this protocol will depend on the monitoring of cells throughout the process.
To this end, follow the volume shown in the table below:

| Bottle size (cm²) | ⅔ of the Volume (mL) |
|---|---|
| 25 | 1.2 |
| 75 | 5.3 |
| 175 | 16.6 |

Below is a breakdown of this process:
In a biological safety cabinet, remove the bottle lid and put it to the side, facing up.
Choose the method that will be used for the aspiration of the supernatant medium, that is, medium change with suction equipment or medium change with pipette.
Turn the bottle so that the solution is on the opposite side of the cell growth.
Perform the aspiration gently, of a volume corresponding to ⅔ of the total.
Gently dispense the appropriate volume of pre-heated medium, to the wall opposite to the cell growth surface.
Close the bottle and spread the medium all over the surface.
Check in the map the sample incubation position,
Put it in place and if the lid of the flask does not have a filter, unsrew the lid leaving it half open.

Total Medium Volume Change:

Usually this exchange is performed during the sample passages, when the cells are already adapted to the medium and in the log phase of multiplication. Whether or not to perform this item will depend on the monitoring of the cells throughout the process. Below is a table with the appropriate volume of solution B and Culture Medium.

Volume of Solution B

| Bottle size (cm²) | Total volume (mL) |
|---|---|
| 25 | 2 |
| 75 | 5 |
| 175 | 10 |

Culture Medium volume:

| Bottle size (cm²) | Total volume (mL) |
|---|---|
| 25 | 2 |
| 75 | 8 |
| 175 | 25 |

Below is a breakdown of this process:
In a biological safety cabinet, remove the bottle lid and put it to the side, facing up.
Choose the method that will be used for the aspiration of the supernatant medium.
Turn the bottle so that the solution is on the opposite side of the cell growth.
Gently perform aspiration of the entire bottle volume.
Dispense the appropriate volume of Solution B, according to table above, on the side opposite to the growth of cells.
Invert the bottle and homogenize it with gentle movements, causing the liquid to spread across the entire lower surface of the bottle, clearing the monolayer.
With the aid of the suction tip or pipette, remove the “dirty” Solution B.
If there are still cellular debris, perform the cleaning again.
Smoothly dispense the appropriate volume of pre-heated medium, opposite to the cell growth surface.
Close the bottle and spread the medium all over the surface.
Check in the map the sample incubation position.
Put it in place and if the lid of the bottle does not have a filter, unscrew the lid leaving it half open.

Traceability of Information

Each step of cell tracking is recorded in detail in the following record.

Tracking Record:

All media change are recorded in the Medium Change Record.

Medium Change Record.

This records include all unplanned occurrences and requirements not conforming to the previous specifications. Any record generated in the processing of the sample is filed in the BENEFICIARY DOSSIER in the Quality Control department.

Cell Adhesion

Cell Adhesion is an extremely important analysis for the continuity of the process, since the stem cells of the pulp of the deciduous tooth begin to multiply only after this stage.

Procedure

Preparation for Cell Adhesion Verification:

The preparation for carrying out the cell adhesion check consists of checking the equipment and the software necessary, as described below.

Inverted Microscope

The inverted microscope will be used to locate the image that will prove cell adhesion.

To do this, proceed as follows:
Clean the microscope table with cloth soaked in alcohol 70° GL.
Turn on the equipment by turning the button located on the lower right side of the microscope.
Adjust the light intensity on the button on the right side of the bottom of the equipment.

Software

The software that will be used to capture the image will depend on the equipment that will be used for this activity.
Take the image capture and save it to an electronic file as described in the procedure below.

Procedure

The cell adhesion verification procedure consists of the photographic record, at two random points in the bottle, of cells with morphological characteristics described in FIGS. 16 to 18.

This procedure is performed within a maximum of 24 days after processing, so if cell adhesion is not verified within this period, notify the person in charge of the area and record the occurrence on the Non-Adherent Cells Communication form, above, for appropriate measures.

This procedure is directly linked to the Monitoring and Medium Change item.

The sample should not remain for more than 5 minutes under observation under a microscope. If this time is exceeded, return the sample to the incubator and leave it there for at least 10 minutes.
For cell adhesion verification, proceed as described below.
Clean the edges of the oven with cloth soaked in 70° GL alcohol.
Check where the sample is located using the map of the incubator.
Open the incubator, close the lid of the bottle and take it carefully to the table of the microscope.
With cloth soaked in alcohol 70° GL, clean the bottom of the bottle and place it on the microscope table, with the lid facing the left side of the collaborator.
Adjust the focus by moving the macro and micrometer adjustment of the microscope.
Check the presence of fusiform and scattered cells.
Perform the image capture according to the specific software of the equipment used.
Repeat this procedure again until two random points are verified in the bottle.
DO NOT exceed 5 minutes in observation.
Enter the patient's electronic folder identified with the patient ID.
Within this folder, if it is the first image to be registered, create a folder with the beneficiary ID, for example: 001.1 and within it, a folder of Monitoring.
Within the Monitoring folder, create a folder identified with the number of days the sample is incubated (i.e., Day 5).
In this folder, identify the photo in the File Name field as the example: ID—Incubation Days—Cell Feature.
In the Type field, select the relevant JPEG format of file and click Save.

Traceability of Information

Record the performing of the Cell Adhesion as described below:
Copy the saved image in the path quoted above and paste it into the Cell Adhesion Record, found in the master list of records.
Paste the image into Table 1; adjust it if necessary using the tools of an appropriate text editor or system.
Repeat this procedure with the other image.
Print the document and attach to the forms in progress.

It there is no cell adhesion in up to 24 days, fill out the Non-Adherent Cells communication form, presented above, proceeding according to the item above, but with images proving the non-adherent cells.

Cell Expansion

Cellular expansion or cell passage usually occurs after 24 days, when cells are already attached and begin to multiply.

### Definitions and Abbreviations

Confluence: Decreased spaces between cells with formation of a layer or carpet of cells due to the multiplication and increase of the number of cells in a culture flask.
Debris: Cell waste.
Basal medium: RPMI medium, DMEM or DPSCBM. i.e., culture media used for cell growth without the addition of other components.

Procedure

Preparation for Expansion or Passage of the Sample:

Inanimate Materials:
suction hoses kit;
1000, 200 and 20 μL micropipettes;
1.0, 0.2 ml sterile tips with and without filter;
05 10 ml sterile serological pipettes;
07 sterile Pasteur pipettes;
02 Eppendorf tubes (non-sterile for cell counting);
03 15 ml conical bottom tubes or appropriate tube for sterile centrifugation;
02 25 cm² culture bottles;
02 75 or 175 cm² culture bottles (in case post-split expansion is made),
labels for identification of the flasks.
sterile wipers

Solutions:
Xeno-free Medium preheated to 37° C.;
Basal medium preheated to 37° C.;
Solution B (1×PBS, Penicillin, Streptomycin) previously heated to 37° C.;
Tryple solution for cell detachment;
0.4% Trypan Blue Solution (If counting and viability are being done). Warning: Toxic

Equipment Verification

All equipment that will be part of the process shall be checked prior to sample processing for proper operation, cleaning and general maintenance. Below are listed the equipment that will be part of the process.
Biological safety cabinet;
Water bath;
Centrifuge;
CO2 incubator;
Semi-automatic pipette;
Stopwatch;
Inverted Microscope;
Automatic Cell Counter,
Neubauer chamber and coverslip;
Countess counting chamber.

Cell Expansion or Passage

The cell expansion or passage procedure occurs when the samples reach adequate confluence.

The sample after processing corresponds to passage zero, that is, the cells that grew were in the pulp and have not yet been transferred to a new bottle.

The bottles and tubes that will be used for the passage of a sample must be IDENTIFIED before the process.

Methodology for Removal of the Medium

Removal of the Medium with Suction Equipment:

The suction hose kit is packaged in two parts, one containing only the hose and the other containing the hose, the quick coupling and the tip connection. The kit is supplied to the processing sector in appropriate and sterile package.
Insert the packaging with the hose, the quick coupler and the tip connection in a biological safety cabinet.
Open the other package in the external area, insert one end hose in the suction equipment and the other end in the biological safety cabinet.
Open the other package, containing the silicone hose, the suction tip and the quick coupling and connect the hose attached to the equipment.
Inside the cabinet, turn on the suction.
With the aid of the suction tip, connect the 200 μL sterile pipette tip.
Open the bottle and insert the suction tip deep enough to remove the medium from the bottle.
Proceed with the protocols of passage described below.

Medium Change with Pipette:
Open the bottle, turn it so that the medium is on the opposite side of the growth.
Insert the pipette to the bottom of the bottle and remove the medium.
Disposal the medium appropriately.
Proceed with the protocols of passage described below.

Passage for Cell Growth Balance

After processing the sample and verifying its growth, it is possible to observe an inhomogeneous growth of the cells, that is, to find some points with great growth of cells from a CFU, but several other points without the growth of cells. If the passage is made to two 25 cm² bottles, the cell density may not be sufficient for cell growth. Thus, a passage is made to another 25 cm² bottle in order to balance the growth of these and prevent the senescence of the cells. For this procedure, carry out the passage normally as described below, but inoculate the cells in a single 25 cm² bottle.

Split for Quality Control and Cryopreservation (First Passage)

When the growth of the post-processing or post-cell balance sample reaches adequate confluence, the Quality Control Split is performed, in which the suspension of this growth is divided and transferred to two 25 cm² bottles.

The expansions are carried out in the two bottles, in the same way, that is, a bottle for Quality Control and a bottle for Cryopreservation. The two bottles of the Split (CRYO and Q.C.) are worked on using the same materials and solutions at the same time. To do this, proceed as described below:
Perform the photographic recording of the sample.
Print the labels to identify the bottles being 09 for the centrifuge tube (Cells), 01 for the bottle of CRYO, 01 for the bottle of Q.C., 01 for the map of the incubator, and identify them.
In a biological safety cabinet, remove the cap from the bottle and place it on the side, facing upwards.
Choose the method that will be used for the aspiration of the supernatant medium.
Perform the aspiration, smoothly, of the whole volume of the bottle.
Dispense 2 mL of Solution B on the opposite side of the growth of the cells.
Turn the bottle and homogenize with gentle movements, causing the liquid to spread throughout the lower surface of the bottle, cleaning the monolayer.
With the aid of the suction tip or pipette, remove the “dirty” Solution B.
If cell debris is still present, perform the cleaning again and repeat the process.
Add 1.5 mL (1 vial) of cell detachment solution and spread it over the entire growth surface.
Incubate for 10 to 30 minutes at a temperature of 37° C. and 5% CO2.
After this period, check in microscope if there was complete cell detachment, that is, refringent, circular, suspended cells.
If the cells persist without fully detaching, repeat the incubation for ±5 minutes until complete detachment.
Add 3.0 mL of basal culture medium gently to the cell growth surface and homogenize the bottle by suctioning and dispersing the suspension into the bottle.
Remove all contents of the bottle with the aid of a serological pipette and dispense in an sterile, previously identified appropriate tube.
Centrifuge for 5 minutes at 1000 rpm or 178G.
Remove the tube and dispense the supernatant, inverting the tube.
Add 4 mL of preheated Xeno-free medium, resuspend the pellet and homogenize.
Transfer an aliquot of 2 mL to a bottle identified with unequivocal numbering, 1^{st} passage and the initials CRYO, which will be expanded to cryopreservation.
Transfer a 2 mL aliquot to a bottle identified with unequivocal numbering, 1^{st} passage and Q.C., which will be expanded for Quality Control analysis.
Incubate the bottles with a temperature of 37° C. and atmosphere of 5% CO2, superimposed one on the top of the other.

Post-Split Cellular Expansion (25 to 75 or 75 to 175)

Cellular expansion is performed to provide sufficient cell density for cryopreservation.

This step is performed after the first passage. Each time the cells are transferred to another bottle, we sequentially number a new passage of the cells. To perform the passages, proceed as described below:
Perform the photographic recording of the sample.
Print the labels to identify the bottles being 01 for the centrifuge tube (Cells), 01 for the bottle of CRYO, 01 for the bottle of Q.C., 01 for the map of the incubator, and identify them.
I a biological safety cabinet, remove the cap from the bottle and place it on the side, facing upwards.
Choose the method that will be used for the aspiration of the supernatant medium.
Gently aspirate the entire volume of the bottle and introduce as little of the pipette/tip as possible into the bottle.
Perform the same procedure with the other sample.
Dispense the appropriate volume, according to the table below, of Solution B on the opposite side to the growth of the cells in the two bottles.

Volume of the Solution B

| Size of the bottle (cm²) | Total volume (mL) |
|---|---|
| 25 | 2.0 |
| 75 | 5.0 |

Turn the bottles and homogenize them with gentle movements, causing the liquid to spread throughout the lower surface of the bottle, cleaning the cell growth monolayer.
With the aid of the suction tip or pipette, remove the “dirty” Solution B.
If there is still cellular debris, perform the cleaning again and repeat the process.
Add adequate volume of cellular detachment solution, according to the table below:

Detachment Solution

| Size of the bottle (cm²) | Total volume (mL) |
|---|---|
| 25 | 1.5 (01 Vial) |
| 75 | 4.5 (03 Vial) |

Incubate the bottles for 10 to 30 minutes at 37° C. and 5% CO2.
After this period, check in microscope if there was complete cell detachment, that is, refringent, circular, suspended cells.
If the cells persist without fully detaching, repeat the incubation for 25 minutes until complete detachment.
Add appropriate volume of basal culture medium gently on the cell growth surface and homogenize the bottle by suctioning and dispersing the suspension into the bottle.

Basal Medium

| Size of the bottle (cm²) | Total Volume (mL) |
|---|---|
| 25 | 3.0 |
| 75 | 8.0 |

Remove all contents of the bottle with the aid of a serological pipette and dispense it in a sterile, previously identified appropriate tube.
Centrifuge for 5 minutes at 1000 rpm or 178G.
Remove the tube and dispense the supernatant, inverting the tube.
Add 4 mL of preheated Xeno-free medium, as per table below, to both tubes to resuspend the pellet and homogenize them.
If necessary, carry out the counting and viability of the cells according to specific procedure, taking 50 μL of this suspension.
Transfer the entire volume of the suspension of the tube identified as CRYO to a bottle also identified by the symbol CRYO, which will be expanded for cryopreservation.
Transfer the entire volume of the tube suspension identified as Q.C. for a bottle also identified with the acronym Q.C., which will be expanded for Quality Control analysis.
Complete the volume of the culture bottle according to the table below, with Xeno-free culture medium.

Xeno-free medium

| Size of the bottle (cm²) | Total Volume (mL) |
|---|---|
| 25 | 2.0 |
| 75 | 8.0 |
| 175 | 25 |

Incubate the bottles with a temperature of 37° C. and an atmosphere of 5% CO2, superimposed one on the top of the other.

Traceability of Information

Each expansion of the processing is recorded in the following Cellular Expansion Record:

Cellular Expansion Record:

This record includes all unplanned occurrences and requirements not conforming to the specifications.

Any record generated in the processing of the samples is filed in the BENEFICIARY DOSSIER in the Quality Control department.

Cryopreservation

The cryopreservation procedure of the biological samples is the last step that occurs with the beneficiary cells, in which the samples are subjected to a gradual freezing until reaching a temperature of −120° C. and transferred to the liquid nitrogen tank.

### Definitions and Abbreviations

Confluence: Decreased spaces between cells with formation of a layer or carpet of cells due to multiplication and increase of the number of cells in a culture flask.
Debris: cellular waste.
Quarantine: Sample in a specific place and NOT released for use.
DMSO: Dimethyl Sulfoxide.
PBS: Phosphate Buffer Solution.

Procedure

Preparation for Expansion or Passage of the Sample:

Inanimate Materials:
Suction hoses kit;
1000, 200 and 20 μL micropipettes;
1.0, 0.2 mL sterile tips with and without filter,
12 10 mL sterile serological pipettes;
04 sterile Pasteur pipettes;
02 Eppendorf tubes (For Cell Counting);
02 15 mL conical bottom tubes or appropriate tube for sterile centrifugation;
03 50 mL conical bottom tubes;
10 Cryotubes,
Labels for identification of the tubes.

Solutions:
Cryopreservation medium;
Culture medium for inactivation (Xeno-free basal medium);
Solution B (1×PBS, Penicillin, Streptomycin) preheated to 37° C.;
DMSO (previously sterilized by filtration);
Solution for cellular detachment;
1×PBS at room temperature;
Trypan Blue solution 0.4%. Warning: Toxic.

Equipment Verification

All equipment that will be part of the process should be checked before the sample processing for proper operation, cleaning and general maintenance. Below are listed the equipment that will be part of the process.
Biological safety cabinet;
Water bath with stirrer,
Centrifuge;
CO2 Incubator;
semi-automatic pipette;
inverted microscope;
automatic cell counter (Countess);
Neubauer Chamber and cover slip,
Stopwatch,
Freezal.

Process of Cryopreservation

Cryopreservation is the storage of samples at low temperatures, in order to preserve cell viability for future applications. The freezing is performed in specific equipment allowing gradual temperature decrease, thereby maintaining ideal conditions for maintaining viability. FIG. 23 indicates the temperature ramps used in the process.

Sample cryopreservation occurs when the samples are in the final expansion step and already confluent and in suitable cell density, as shown above.

Cryopreservation is performed in approximately 07 cryotubes, of which:
04: Samples for the beneficiary with 1.5 mL per tube.
03: Samples for Quality Control with 1.0 mL per tube.

The cryopreserved samples retain the passages in which they are, i.e., the passage in which appropriate cell density has been achieved.

Criteria for Cryopreservation of the Sample

Samples with potential for cryopreservation shall meet the following criteria:

1. Possess the ability to adhere and multiply to plastic.
2. Density of cells>1×10⁶ cells/Viable Cryotubes (Live cells), for the CRYO bottle.

If the above criteria are met, proceed according to Item Cryopreservation of the Samples.

If the criteria are NOT met, proceed as described below referring to the numbering of each criterion:

1. Inform the Quality Control to carry out a new sample collection and complete form PO.02.01.021.C: Sample Rejection Record, if the duplicate or any deviation from the process, which is not described above, is not sufficient to meet the criteria.
2. Add the solutions in a single tube and resuspend in a tube with 9 mL of Xeno-free medium. Transfer 3 mL to 3 175 cm bottles and return to incubation or transfer the suspension to larger volume bottle and subject to new growth.
The cell density verification is performed by passing a tube with 0.05 mL of the CRYO bottle suspension for Quality Control, which will OK for the cells to be cryopreserved.

Cryopreservation of the Samples

The initial methodology used for cryopreservation is the same used for the passage or cell expansion. At the end of the process, the number of cells required is transferred to appropriate tubes, in specific medium, after which happens the addition of cryoprotectant solution.

The same procedure performed for the bottle identified and CRYO is also performed for the bottle identified as Q.C.

In the process of storing the sample for quarantine, attach the guard registration form generated by CoolBase© software, or file the electronic record in the specific folder of the beneficiary, indicating the specific location where the cells will be cryopreserved.

To perform the cryopreservation procedure, proceed as described below:
Issue the identification labels for the tubes, containing ID and sample passage for Cryopreservation at a minimum. For quality controls, proceed as described in the items below or in the specific procedures.
   2 for the centrifuge tubes (CRYO and Q.C.).
   4 for the Cryopreservation tubes (CRYO).
   4 for the Quality Control tubes (Q.C.).
   2 for Karyotype Assay, the sample ID being Karyotype I and Karyotype II.
   I for the immunophenotyping test, with the sample ID and identification of the Immunof. analysis.
   1 for the osteogenic differentiation assay, with the sample ID and the identification of the Ost. Dif. analysis.
   2 for the sterility test, with the sample ID and the start date of the analysis.
   1 for counting and viability, with the sample ID.
Perform the photographic record of the samples, according to the procedure.
I a biological safety cabinet, remove the caps from the bottles and place them on the side, facing upwards.
Choose the method that will be used for the aspiration of the supernatant medium.
Gently perform the aspiration of the entire volume of the bottle.
Carry out the same procedure with the other sample and dispense the contents in an appropriate vial of discard.
Dispense 10 mL of Solution B on the opposite side to the growth of the cells in each of the two bottles.
Turn the bottles and homogenize them with gentle movements, causing the liquid to spread throughout the lower surface of the bottle, cleaning the cell growth monolayer.
With the aid of the suction tip or pipette, remove the “dirty” Solution B.
If cellular Debris is still verified, perform the cleaning again.
Add 10.5 mL of detachment solution into each of the two bottles.
Incubate the bottles for 5 to 20 minutes at 37° C.
Remove the bottle from the incubator and, using gentle movements, tilt the bottle to check the cellular detachment observing the turbidity of the suspension.
If necessary, check under microscope if there was complete cell detachment, i.e., presence of refringent, circular, suspended cells.
If after this process the cells persist without fully detaching, repeat the incubation for ±5 minutes, until complete detachment.
Add 4 mL of inactivation medium (Basal Medium) gently to the cell growth surface and homogenize by suctioning and dispensing the suspension into the bottle.
Remove all contents of the bottle with the aid of a serological pipette and dispense into a sterile conical tube suitable for centrifugation, previously identified.
Centrifuge for 5 minutes at 1000 rpm or 178G.
Remove the tube and dispense the supernatant, inverting the tube.
Add 5 mL of 1×PBS to resuspend the pellet and homogenize them.
Centrifuge for 5 minutes at 1000 rpm or 178G.
Remove the tube and dispense the supernatant, inverting the tube.
Add 6 mL of cryopreservation medium without the cryoprotectant solution (DMSO) in both tubes (CRYO and Q.C.).
Perform the counting and viability of the cells according to specific procedure, removing 50 μL (0.05 mL) from the suspension of the tube identified as CRYO only.
If the criteria for cryopreservation of the sample are met, identify the appropriate tubes (cryotubes previously sterile) for cryopreservation.
Homogenize the tubes again.
Dispense 1.5 mL of the suspension to cryotubes identified as CRYO.
Dispense 1.0 mL of the suspension into at least 3 cryotubes identified as Q.C., the rest will be added to the quality control tests. Note: If necessary, some tubes may be cryopreserved for further analysis of any Quality Control assay.
Add 0.155 mL of DMSO to the cryotubes identified as CRYO.
Add 0.110 mL of DMSO to the tubes identified as Q.C.
Homogenize the tubes quickly and transfer them to the Cryopreservation sector.
   PRECAUTIONS: After addition of DMSO, the samples should be transferred immediately to the temperature decay equipment.
For the tube identified as Q.C., after cryopreservation of at least 3 tubes proceed as follows:
Identify the bottles and the plates with the labels previously printed.
Dispense 0.1 mL of the suspension into each of the 3 wells of the plate identified as Osteogenic Differentiation.
Dispense 0.5 mL into each of the two bottles identified as Karyotype I and II, I represents one replicate and II the second replicate, which will be used if necessary.
Dispense 0.5 mL into a bottle identified as Immunophenotyping. This analysis can also occur by sending an aliquot directly to the Q.C. sector for analysis in flow cytometry.
Remove 1 mL and dispense 0.5 mL into one vial of Thiogycollate medium and another 0.5 mL into another vial with Casein soy medium for the sterility test.
Proceed according to specific procedures for each test, as well as the records.
Add 6 mL of cryopreservation medium without cryoprotectant solution (DMSO) in the tubes to resuspend the pellet and homogenize them until homogeneous content is verified.
Perform the counting and viability of the cells according to specific procedure, taking 50 μL of each suspension (Q.C. and CRYO).
If the criteria described in Criteria for Cryopreservation of the sample are met, identify the appropriate tubes for cryopreservation.
Homogenize the tubes again.
Dispense 1.5 mL of the suspension into the cryotubes identified as Q.C.
Add 0.155 mL of DMSO to the cryotubes identified as CRYO and 0.110 mL in the cryotubes identified as Q.C.
Homogenize the tubes quickly and transfer them to the Cryopreservation sector.

Precautions

After addition of DMSO, samples should be transferred immediately to temperature decay equipment.

Cryopreservation record is performed on the Cryopreservation of the Sample Record.

Cryopreservation of the Sample Record:

Quarantine

Quarantine is the time the sample is stored, as per item above, until the results of Quality Control meet the item Criteria for Acceptance of Cryopreserved Samples. After this period, the samples are released for use when requested.

Quality Control

In the Quality Control sector, the tests that endorse the cryopreservation of the cells are carried out, that is, the cryotubes that are cryopreserved in a Quarantine tank are transferred to the tank of released samples. Next, the analyses carried out and the details of the process will be shown.

Sterility Test

The procedure of sterility of biological samples proves that there was no contamination in the sample during the handling of the sample throughout the process.

Procedure

The sterility test is performed to certify that the samples to be cryopreserved are sterile, i.e., free of microorganisms. The test is performed in a laminar flow hood and in a controlled environment.

The aliquots of the biological samples are derived from the growth and expansion of cells that will undergo cryopreservation, that is, from the culture bottle identified as CRYO. They are collected according to item Cryopreservation of the samples.

Preparation for Sterility Test

In order to facilitate the preparation, below are the materials needed for each stage:

1—Inanimate materials:
1.0 ml tip with sterile filter;
sterile Pasteur pipette,
appropriate cloth, sterile;

### 2—Solutions:

Soybean casein digest broth,
Thioglycolate Broth Medium;
Samples for testing.

### 3—Equipment:

200 and 20 μL micropipettes;
Laminar Flow hood;
Bacteriological Incubator with temperature set at 25±2° C.;
Bacteriological Incubator with temperature set at 35±2° C.

Precautions for Sterility Testing by Direct Inoculum Method

The sterility test is performed according to the American Pharmacopoeia USP 71—Sterility Test, Direct inoculation. Below are the recommendations and precautions for the test:
The test should be performed only by trained and qualified personnel.
Tests should be carried out under aseptic conditions in laminar flow hood, which must be installed in clean room class B-ISO 7.
The conditions should be adequate to prevent accidental contamination of the sample during the test and also not affect the detection of possible contaminants.
Print labels for identification of the tubes where the tests will be carried out.

Sterility Testing

The test is performed using 02 samples, one inoculated in soybean casein digest broth medium and the other inoculated in thioglycolate broth medium. The whole procedure is performed in laminar flow hood and using aseptic techniques. After performing the preparations described in the above items, carry out the test as follows:
Label the tubes of culture medium, soybean casein digest broth and thioglycolate broth.
Transfer aliquots of the test into the laminar flow hood.
Perform external asepsis of the samples with suitable cloth soaked in alcohol 70° GL.
Leave the tubes with medium and with samples half-screwed.
With the aid of a Pasteur pipette or pipette with sterile tip filter, transfer the contents of the test tube to the tube containing culture medium.
Perform the same procedure for the second sample, inoculating the content in another tube containing culture medium.
Incubate the tube containing soybean casein digest broth culture medium in bacteriological incubator at 25±2° C.
Incubate the tube containing the thioglycolate broth culture medium in a bacteriological incubator at 35±2° C.
Monitor daily for 14 days.

Interpretation of Results

The interpretation of the results is made by inspecting the culture medium tubes. Tubes are monitored daily for 14 days as described below:
Inspect the media for macroscopic evidence of microbial growth (turbidity).
If at the end of the incubation period, there is no evidence of microbial growth, the sample under examination meets the sterility requirement
If growth of microorganisms is observed the sample does not meet the sterility requirement, unless there is another source of failure during the test run, for example, unrelated contamination.
The sterility test may be considered invalid if one or more of the following conditions are met:
   a) The microbiological monitoring data of the area where testing was performed reveals failure.
   b) The review of the analytical procedures used during the test reveals failure.
   c) Microbial growth is observed in the negative controls.
   d) After identification of the microorganism(s) isolated from the test, the growth of this species can be attributed unequivocally to failures related to the material used and/or the techniques used in the sterility test.
If invalid, the sterility test should be repeated with the some number of units of the initial test.
If, after repeating the test, is not observed microbial growth, the sample meets the sterility requirement.
If it is observed microbial growth after repeat testing, the sample under examination does not comply with the sterility requirement.
Conventional microbiological/biochemical techniques are generally satisfactory for identification of microorganisms recovered in a sterility test.
In case of considering only that, after determining the identity of the microorganisms isolated in the test, the growth of this (these) species can be unequivocally attributed to failures regarding the material and/or technique used in the sterility assay procedure may be necessary to employ more sensitive techniques to demonstrate that the isolated micro-organism in the product is identical to the one isolated from the materials or the environment.
If the routine microbiological/biochemical identification techniques can demonstrate that 2 isolates are not identical, these methods may not be sufficiently sensitive or reliable to provide unambiguous evidence that two isolates are derived from the same source
Molecular methods can be used to determine if two microorganisms originated from the same clone and have common origin

Note: the microorganism identification tests are performed by a third party. If there is sample contamination, the contaminated tubes are sent to the decontamination sector. Records

Records are made on specific forms and as described below:
Complete the results of the monitoring in the Sterility Record.

Sterility Record:
In the field Status, fill C to Compliant, that is, the medium does not present apparent turbidity and NC to Non-compliant, if there is turbidity in the medium indicative of microorganisms growth.

Note: If there is growth of microorganisms, perform the test again, with the number of samples taken at the beginning. If the growth of microorganisms is not verified, the test complies with the sterility requirement.

Cell Counting and Cell Viability

The counting and viability procedure is performed on the day the sample will be cryopreserved in quarantine, that is, after the procedure described in the item Cryopreservation, an aliquot of the patient sample is withdrawn and submitted to the test.

Procedure

The cell count and viability Is a method that checks the cell density per milliliter (mL) In a given solution, as well as verifies the percentage of dead cells as compared to the living ones, using a dye that allows to distinguish them.

For cell count and viability, it is extremely important to follow the following steps:
Perform the aliquots as described in the Cell Expansion and Cryopreservation of the Samples items.
Make sure the suspension for counting is homogeneous and without visible particulates, in order to avoid a false alarm count.
Aliquot the suspension with a micropipette previously cleaned with sterile cloth soaked in 70° GL alcohol and with a suitable filter tip.
Dispense aliquot of the suspension in an appropriate tube previously identified.

Preparation for Cell Counting and Viability

The aliquot of the suspension for counting and viability is performed in a biological safety cabinet.

The cell counting and viability procedure is carried out on a previously sanitized bench. Below are the materials necessary for the accomplishment of the count and viability.

### 1—Supplies:

0.2 ml tip with and without filter,
04 Eppendorf tubes,
Marker to identify the sample.

### 2—Solutions:

Cell suspension;
0.4% Trypan Blue Solution (Warning: Toxic.).

### 3—Equipment:

200 and 20 μL micropipettes;
Biological safety cabinet;
Countess™ counting chamber.

Cell Count and Viability in Neubauer Chamber

Preparation for Cell Count and Viability

The aliquot for cell counting and viability Is performed in a biological safety cabinet.

The cell counting and viability procedure is performed on a bench previously sanitized. Below are the materials needed to carry out the count and viability.

### 1—Supplies:

0.2 ml tip with and without filter,
04 Eppendorf tubes,
Marker to identify the sample;
Appropriate cloth.

### 2—Solutions:

Cell suspension;
0.4% Trypan Blue Solution (Warning, Toxic.)

### 3—Equipment:

200 and 20 μL micropipettes;
Biological safety cabinet;
Neubauer Chamber;
Coverslip,
Microscope.

Overview of the Neubauer Chamber:

FIGS. 03 show the sample insertion chambers (1), FIG. 04 shows the location of the remaining sample (3), the sample (4), the blade (5) and counting grid (2). And as an illustration, FIG. 05 shows the coverslips box (6) and Coverslips (5).

Cell Count and Viability

After the completion of the suspension collection in a biological safety cabinet, proceed as follows:
Label an Eppendorf tube with the beneficiary ID and 1:10 dilution.
Transfer 10 μL of 0.4% Trypan Blue solution to this tube.
Homogenize the tube with the suspension aliquot in cabinet.
Transfer 10 μL of this suspension to a tube containing 10 μL of 0.4% trypan blue.
Homogenize.
Attach the cover slip (5) on entry of the sample (4) (FIG. 06).
Transfer 10 μL of this solution to one of the inputs (A or B) of the Neubauer counting chamber, as shown in FIG. 07.
Transfer the chamber to the microscope table.
Adjust the lens for 10 or 20×.
Focus the image in the squares, as shown in FIG. 08.
Squares are divided into 04 parts, as follows: UL—Upper Left; UR—Upper Right; LL—Lower Left and LR—Lower Right.
Using the Cell Counting and Viability form, perform the counting as in FIG. 09.
Perform the count of 04 squares in the same manner described above.
Count the refringent cells as ALIVE and stained cells in Blue and not refringent as DEAD.
Record the results in specific form.

Observations: If the cell count does not reach or exceed the RANGE of the method, i.e., a value outside of 2.5×10⁵ to 2.5×10⁶ cells per ml, proceed as follows:
Concentrate the sample by centrifugation when less than 2.5×10⁵.
Dilute the sample by addition of the same solution of the original suspension when more than 2.5×10⁶ and multiply the final result by the dilution performed.
When the suspension is counted in the dilution 1:1, multiply the result by 2.

Preparation of Cell Differentiation Media

The preparation of the Osteogenic Cell Differentiation medium is performed in a way to facilitate its use and storage, as well as to segregate the medium that will be used in each sample, guaranteeing the traceability of the process.

Procedure

Supplies, Solutions and Equipment:
Culture Medium for Genetic Control;
1.25 Dihydroxyvitamin D3;
Dexamethasone;
Ascorbate—2-Phosphate;
B Glycerophosphate;
Pasteur pipette;
Serological pipette;
Sterile tips;
Micropipette;
Volumetric balloon;
Biological safety cabinet.

Cautions and Precautions:
The entire procedure for handling solutions and reagents is carried out within the Biological Safety Cabinet using aseptic techniques.
Use masks and gloves during the preparation of the solutions and discard them when another solution is prepared in order to avoid contamination between the reagents.

Preparation of the Medium for Osteogenic Differentiation

Separate the following reagents to prepare the solution:
Culture medium for Genetic Control GCM;
1,25 Dihydroxyvitamin D3;
Ascorbate-2-Phosphate;
β Glycerophosphate.

In principle, each solution is prepared separately and fractionated into specific, sterile tubes labelled with the compound name, brand and expiration date. After this procedure they are stored as described in the preparation.

To carry out the preparation of each reagent, proceed as described below:

Preparation of 1,25 Dihydroxyvitanin D3:

The final concentration of this compound is 0.01 μM and Its molecular weight is 416.64 g. The final volume of culture medium that will be prepared is 30 mL, so carry out the following procedure:
In a biological safety cabinet perform the external asepsis of the flask.
Open the flask with care.
Dispense 1 mL of Basal Culture Medium.
Homogenize in vortex.
Fraction 14 μL in sterile tube and label them.
Store in a freezer at ≤−10° C.

Preparation of Ascorbate-2-Phosphate:

The final concentration of this compound is 50 μM and its molecular weight is 289.54 g. The final volume of culture medium that will be prepared is 30 mL, so carry out the following procedure:
Weight 0.45 g of the reagent in an analytical balance and transfer to a 10 mL flask.
Complete the volume with Basal Culture Medium.
Homogenize in a vortex.
In a biological safety cabinet, carry out the filtration in a 0.22 μm filter, dispensing in a sterile container.
Dispense 0.9 mL of Basal Culture Medium into sterile tube.
Remove aliquots of 0.1 mL and dispense into each tube containing 0.9 mL of Basal Culture Medium and label them.
Store in a freezer at ≤−10° C.

Preparation of β Glycerophosphate:

The final concentration of this compound is 10 mM and its molecular weight is 216.04 g. The final volume of culture medium that will be prepared Is 30 mL, so carry out the following procedure:
Weight 6.5 g of reagent in an analytical balance and transfer to a 100 mL flask.
Complete the volume with Basal Culture Medium.
Homogenize in vortex.
In a biological safety cabinet, carry out the filtration in a 0.22 μm filter, dispensing in a sterile container.
Remove aliquots of 1.0 mL, and dispense into sterile tube and label them.
Store in a freezer at ≤−10° C.

Preparation of Osteogenic Culture Medium

The osteogenic culture medium is composed of the solutions prepared above, and they are frozen after preparation and thawed only at the beginning of the differentiation. The entire contents of this vial are diluted in 30 mL of GCM medium. The preparation of this medium is done according to the Osteogenic Medium Record form. Following the form is an overview of the preparation.

Osteogenic Medium Record Form:
Defrost the three compounds prepared above.
With the aid of a Pasteur pipette of micropipette with sterile tip, remove the solution from the tube and dispense in an appropriate volume of medium.
Homogenize, cover with foil and store in a refrigerator.
The medium is valid until the end of the process, that is, 30 days after preparation.

Osteogenic Cell Differentiation

The procedure of Osteogenic Cell Differentiation aims to characterize one of the characteristic plasticity of the stem cell of the deciduous tooth pulp.

Procedure

Supplies, Solutions and Equipment:
Cell suspension;
GCM Medium;
Osteogenic Differentiation Medium;
Alizarin Red Dye Solution;
4% Formaldehyde Fixing Solution;
Ultra-purified water,
Sterile 12-well Culture Plate;
Pasteur pipette;
Micropipette;
Microscope;
Centrifuge.

Cautions and Precautions:
Identify a plate with the beneficiary ID and the differentiation that is taking place, on the lid and also on the side, so that there is no loss of traceability.
The entire sample handling procedure is performed within the biological safety cabinet using aseptic techniques.
During the medium exchange, take care to ensure that the monolayer is not disturbed.
Never use the same tip in the positive control wells (PC) and in the well with the negative control sample (NC).
Always check the plate map to identify the samples.
Be careful not to put Differentiation Medium in Negative Control.
The Negative Control will always be the last well of the triplicate.

Osteogenic Differentiation

Osteogenic differentiation is performed to verify the differentiation potential of stem cells from the baby tooth pulp in bone cells. These undifferentiated cells do not deposit extracellular calcium, this deposit being indicative of the differentiation of stem cells into osteoblastle cells. The procedure is divided in three stages, the first being the sample inoculation, the induction of differentiation and the staining of the calcium deposits.

To differentiate, proceed as described below.

Sample Inoculation:
Inoculate 1×10 cells/well in a 12-well plate in triplicate.
Add 2 mL of GCM Medium to each well.
Incubate the sample at 37° C. and 5% CO2.
Allow the sample to incubate for 48 hours or until confluency>90% is achieved.
If you need to change medium, use the Medium Change record below and attach it to the documentation.

Medium Change Record:

Induction of Differentiation

After the confluence of the wells reaches the appropriate percentage, perform the following procedure:
Remove the medium carefully with a micropipette.
Dispense 2 mL of Medium for Osteogenic Differentiation smoothly, by the edge of the well.
Use one tip for each well.
Dispense 2 mL of Initial Medium for Quality Control into the well of the negative control sample, in the same manner as above.
Incubate the sample again.
Perform this procedure between 3 to 4 days.
Repeat this step for 21 days.
Records are made on the Osteogenic Differentiation Analysis Record form.

Osteogenic Differentiation Analysis Record form:

Color Analysis of Calcium Deposits with Alizarin Red:
After 21 days under the condition of differentiation, remove the supernatant medium from the wells and wash once with 1×PBS, 0.5 mL per well.
Remove the PBS and discard.
Fix the cells with 1 mL of 4$ formaldehyde solution for 30 minutes.
After fixation, wash the wells twice with 1 mL of ultra-purified water.
Dispense 1 mL of 2% solution of alizarin red pH 4.2.
Leave to act for 45 minutes in the light.
View in a microscope and capture images for qualitative analysis.

Following the analysis of FIG. 10:
A) Coloration of calcium deposits.
B) Macroscopic view of the plate.

Flow Cytometry

The analysis of cellular immunophenotyping using the flow cytometry technique identifies the specific markers for stem cells of the pulp of the baby tooth, that is, each type of cell has a protein in the cell membrane that characterizes its origin and cellular identity, and after identifying them the purity of the cells that will be cryopreserved is verified.

Procedure

The flow cytometry analysis is used to identify the phenotypic characteristic of stem cells from the pulp of baby teeth. These cells have surface receptors that identify and differentiate them from other types of stem cells.

The analysis is performed using monoclonal antibodies specific for particular receptors. These antibodies are labelled with fluochromophilic molecule, which upon receiving the incidence of specific lasers are excited and emit light. This light passes through specific filters with wavelengths capable of absorbing only the light of interest in a given absorption channel. The captured light is converted into an electronic signal, thereby generating a density plot or a Histogram.

The following procedure describes how to perform and interpret data generated by the system.

For a better understanding of the device and its software, the user manual should be consulted.

In FIG. 11 is an Equipment Overview.

Maintenance and Calibration of the Instrument

Every day and before carrying out the sample analyses, the checks of the solutions used, the cleaning of the SIP and the calibration of the equipment are carried out. Below are the details of this step.

Verification and Preparation of Solutions

Before connecting the equipment, check the following solution flasks:
WASTE: Dispose of contents in an appropriate place according to the waste management plan, and wash it with ultra-purified water.
SHEATH: Add the contents of 01 vial of the Bacteriostatic solution in 01 liter of ultra-purified water. This solution expires 1 month after preparation at room temperature.
CLEANING SOLUTION: Add 3 mL of the contents of 01 vial of Cleaning solution to 197 mL of ultra-purified water. This solution expires after 02 weeks.
DECONTAMINATION SOLUTION: Add the contents of 01 vial of the Bacteriostatic solution to 180 mL of ultra-purified water. This solution expires after 01 month.

Cleaning Sip and System

Cleaning of SIP and system is performed BEFORE starting any procedure, BETWEEN samples and AFTER use of equipment (SHUTDOWN). The probe Is where the sample is drawn and taken to the analysis inside the equipment. The system comprises the fluidic system of the equipment, that is, all the way where there Is the contact of the solutions and sample.

The following procedures are performed on the MANUAL COLLECT tab.

Below are the details of this procedure. Before Procedure:
Click EJECT PLATE.
Place an empty tube on the probe or SIP.
On the main software screen, click BACKFLUSH.
Wait for the procedure to finish.
Place another tube with 2 mL of ultra-purified water in the sample rack, in position A1.
In FILE (Upper left corner) select Open Workspace or Template.
Select the Daily Cleaning Workspace.
In field A01, enter the date of the procedure.
Select the A1 position.
Click Detect Events.
Click RUN or ADD to A01.
At the end of the procedure save the data subscribing it in Workspace itself.
Eject the rack and remove the tube.

Procedure Between the Samples of Beneficiaries and Between the markers of the same sample:
Click EJECT PLATE.
In the left corner of the screen click on WASH.
Proceed with the analyzes again.

If many samples are run on the same workspace, the analyses between the replicates may require homogenization, due to the sedimentation of the cells. This procedure can be performed by removing the tubes and quickly homogenize in vortex or using the AGITATE button on the equipment.

Analysis procedure to shut down the equipment:
Place a tube with 2 mL of ultra-purified water in the sample rack, in position A1.
In FILE (Upper left corner) select Open Workspace or Template.
Select Workspace Water 2′.
In field A01, enter the date of the procedure.
Select the A1 position.
Click Detect Events.
Click RUN or ADD to A01.
At the end of the procedure, save the data, subscribing it in the Workspace.
Eject the rack and remove the tube.
Place the tube with 2 mL decontamination solution, DECONTAMINATION Flask.
In FILE (Upper left corner) select Open Workspace or Template.
Select Workspace Decontamination 2′.
In field A01, enter the date of the procedure.
Select the A1 position.
Click Detect Events.
Click RUN or ADD to A01.
At the end of the procedure, save the data, subscribing it in the Workspace.
Eject the rack and remove the tube.
Place a tube with 2 mL of ultra-purified water in the sample rack, in position A1.
In FILE (Upper left corner) select Open Workspace or Template.
Select the Workspace Water 2′.
In field A01, enter the date of the procedure.
Select the A1 position.
Click Detect Events.
Click RUN or ADD to A01.
At the end of the procedure, save the data, subscribing it in the Workspace.
At the end, the equipment can be switched off.

Laser Calibration

Before the runs of samples that will be carried out on the day, the lasers calibrations are performed. The calibration of these lasers guarantees the alignment and the high resolution of the results.

The equipment has two lasers, which are responsible for the excitation of the fluorochromes.

In FILE open the Lasers Calibration Workspace.

In the 24-tube rack, select the next well from the last analysis performed, i.e., if the previous day the well chosen was A1, select A2 and enter the date of the analysis.

For 8 Peak Beads, select wells A1 to B6 and for 6 Peak Beads, select wells C1 to D6.

When all the wells are used, save a new Workspace by adding the sequential number, referring to the last Workspace, e.g.: Lasers Calibration 2. This procedure is performed to obtain a Control Chart and monitor the equipment over time.

PEAK 8 Blue Laser:
Homogenize the Validation Peak Beads 8 reagent in Vortex.
Add 0.5 mL of purified water to appropriate tube.
Add 2 drops of the Validation Peak Beads 8 reagent to this tube.
Place the tube in the rack.
In the rack selection box, select the field where the sample is located.
Save it as 8 Peak beads and enter the date of the procedure.
Click RUN.
When the number of events is reached, the equipment will stop automatically.

PEAK 6 Red Laser:
Homogenize the Validation Peak Beads 6 reagent in Vortex.
Add 1.0 mL of purified water into appropriate tube.
Add 4 drops of the Validation Peak Beads 6 reagent to this tube.
Place the tube in the rack.
In the rack selection box, select the field where the sample is located.
Save it as 6 Peak beads and enter the date of the procedure.
Click RUN.
When the number of events is reached the equipment will stop automatically. These solutions expire after 01 week and are identified with the name of the solution, the product lot described in the bottle and the expiration date, according the example below:

Analysis of Calibration Results

After the Calibration Beads run, the results are analyzed for confirmation of correct operation of the equipment.

For verification the charts must meet the following criteria:

PEAK 8 Blue Laser
8 peaks in PLOTS, FLI and FLU.
At least 6 peaks in FLIII.
Coefficient of Variation (CV)<5% in the peaks of higher fluorescence intensity, i.e., in the last peaks in all PLOTS.
Enclose population>80% in established Gate, in PLOT FCS/SSC.

PEAK 6 Red Laser:
6 peaks in PLOT FLIV.
Coefficient of Variation (CV)<5% in the peaks of higher fluorescence intensity, i.e., in the last peaks in all PLOTS.
Enclose population>80% in established Gate, in PLOT FCS/SSC.

Record the information in the Lasers Calibration Record and save the Working Space as Lasers Calibration followed by the calibration date.

Lasers Calibration Record:

Analysis of Samples of the Beneficiaries

After the cleaning and calibration procedures, the equipment Is capable of carrying out the phenotypic analysis of the samples of the beneficiaries.

Phenotypic analysis of the samples is performed using 4 markers. CD 105, CD 73, CD 45 and CD 146.

During the cryopreservation process, remove a 0.5 mL aliquot of the tube identified as Q.C. and transfer to a 25 cm² bottle. This process can also occur by sending it directly to the sector.
Wait for a confluence of approximately 70% and highlight the cells according to specific procedure.

Check the reagent label for the channel the label is read on, i.e., FITC on FLI, PE on FLII and etc., thereby verifying that the sample is running on the correct channel.
Send the tube after detachment to the Q.C. sector.

Carry out the analysis as described below:
Centrifuge the tube at 900 RPM or 160G for 5 minutes.
Dispense the supernatant and add 1 mL of Stain Buffer.
Centrifuge the tube at 900 RPM or 160G for 5 minutes.
Dispense the supernatant and add 0.5 mL of Stain Buffer.
Identify 5 tubes as: ID+U; ID+CD 105; ID+CD 73; ID+CD 45 and ID+CD 146.
Dispense 0.1 mL of the cell suspension into 5 tubes, 4 for markers and one for unlabelled cells.
Keep 0.1 mL of Stain buffer to the Unmarked (U) tube.
Add the amount described on the CD 105 reagent label.
Add the quantity described on the CD 73 reagent label.
Add the quantity described on the CD 45 reagent label.
Add the quantity described on the CD 146 reagent label.
Incubate the sample for 30 minutes at room temperature away from light.
At the end of the incubation add 0.5 mL of Stain Buffer to each tube.
Centrifuge at 900 RPM or 160G for 5 minutes.
Dispense the supernatant with the help of pipettes and be careful not to suck the cell pellet.
Resuspend in 0.5 mL Stain Buffer.
Homogenize the sample.
Place the tube in the equipment rack.
Open the Workspace Beneficiary Phenotypic Analysis.
Enter the beneficiary number in the field where the sample was inserted in the rack, according to the identification of the tubes, for the 5 tubes.
Click RUN.
Save the Workspace with the ID of the samples to be run, i.e.: 1111-1, 2222-1 and 3333-1.
For each replicate of each sample and each sample, perform the wash by clicking on WASH.
Wait for all runs to finish.

Analysis of the Generated Data

At the end of the run the data is verified using the generated Histograms. The data verified on the Histograms is on the right side of the line separating the unmarked cells for the positive markers (CD 105, CD 73 and CD 1460, and the left side for the negative marker (CD 45). For each marker there is a Histogram. FIG. 12 shows an example of Histogram generated and the result in percentage.

Analysis Record Form:

The records of each analysis are performed using the Phenotypic Cytometric Analysis form (Analysis Record form above) and the raw data is generated as follows:
On the Desktop screen, create a folder with the beneficiary ID.
In Accuri software, click the analysis histogram with the left mouse button and hold.
Drag it to the bottom right corner of the screen, to show the desktop and sent it to the destination folder.
Open the folder of the beneficiary and insert the plots in the appropriate spaces, according to Phenotypic Analysis by Cytometry (Form 12).
Print the form and fill in the remaining information.

Results and Acceptance Criteria

With the generated data the results are observed.

These results are expressed as a percentage, and this percentage will determine the approval of the phenotypical analysis as described below:

### CD 105:>90%;

### CD 73: >90%;

### CD 146: >60%;

### CD 45: <10%.

The results are completed in a specific form and the raw data generated is attached to it.

If the results of the analyzes are not approved, the sample shall be retested.

Preparation of Solutions for Genetic Control

Preparation of Fixing Solution:

The fixing solution is composed of Methanol (CH3OH) and acetic acid (CH3COOH). Always perform the preparation/handling inside the exhaust hood.

The fixative solution is composed of 3 parts of Methanol and a part of Acetic Acid. For example: 75 mL of Methanol and 25 mL of Acetic Acid, for a 100 mL solution.
Place the reagents inside the exhaust hood.
Collect the fractions of interest, depending on the final volume of the solution, see example above.
Mix the parts in a single tube with lid.
Label it.
This solution expires 01 day after preparation and should be stored in a refrigerator with a temperature of 2 to 8° C.
Register the solution in the Fixing Solution Record.

Fixing Solution Record form:

Hypotonic Solution Preparation

The hypotonic solution Is composed of Potassium Chloride (KCL) dissolved in water. It is used to hypotonize cells.
Weigh the product and transfer to a volumetric balloon or suitable flask.
Add 80% of the final volume in water.
Homogenize until the product is dissolved.
Fill the volume with water.
Label it.
This solution expires after 01 month and should be stored in a refrigerator with a temperature of 2 to 8° C.
Register the solution on the Hypotonic Solution Record form 1.

Hypotonic Solution Record form 1:

Preparation of Banding Buffer Solution

The phosphate buffer solution is composed of Potassium Phosphate Monobasic (KH2PO4) and Sodium Phosphate Dihydrate (Na2HPO4.2H2O) dissolved in water. It is used in the preparation of buffer for banding.
Weigh the product and transfer to a volumetric balloon or suitable flask.
Add 80% of the final volume in water.
Homogenize until the product is dissolved.
Fill the volume with water.
Label it.
This solution expires after 01 month and should be stored in a refrigerator at a temperature of 2 to 8° C.
Register the solution on the Hypotonic Solution Record Form 2.

Hypotonic Solution Record Form 2:

Preparation of Banding Solution

The Banding solution Is composed of trypsin (enzyme) and buffer solution. It Is used in the process of chromosome digestion.
Weigh the product and transfer to a volumetric balloon or suitable flask.
Add 80% of the final volume of buffer solution.
Homogenize until the product is dissolved.
Fill the volume with the same solution.
Label it.
The solution expires after 1 day and should be stored in a refrigerator at a temperature of 2 to 8° C.
Register the solution in the Banding Solution Record Form.

Banding Solution Record Form:

Preparation of Coloring Solution

The Coloring solution Is composed of Methylene Blue Eosin (dye) and buffer solution. It is used in the process of chromosome staining.
Fraction the buffer solution in a capped flask.
Add Giemsa to the flask.
Label it.
This solution expires after 01 day and should be stored in a refrigerator at a temperature of 2 to 8° C.
Register the solution on the Coloring Solution Record Form.

Coloring Solution Record Form

Genetic Control

Karyotype analysis of the processed samples is performed with the aim of verifying the chromosomal stability after the process of cell proliferation.

Procedure

The analysis of genetic control in stem cells is important, since cells can acquire chromosomal changes during the process of adaptation to the culture which may become permanent, increasing cellular tumorigenicity. Below is the complete protocol guaranteeing the quality standards of this company. The occurrence of tumorigenesis is an important obstacle in the viability of the use of stem cells in future clinical treatments.

Dental pulp stem cells, after extraction and proliferation in specific culture medium, are extracted and cultured in a medium supplemented for genetic control, which will be performed using classical cytogenetic analysis (GTG banding), standard protocol and widely used to check chromosome integrity.

This procedure allows the identification of chromosomal, numerical and structural changes in the resolution of 5 to 10 Mb. Chromosome number alteration is a numerical variation of chromosome constitution, such as: Euploidia (alteration of chromosome set (n) Aneuploidy (loss or gain of one or more whole chromosomes). The structural chromosomal alterations are from losses, gains or chromosomal reorganization. This type of change can be classified into: Deletion, duplication, inversion, translocation and ring chromosome. All chromosomal change identified is described according to the International System for Human Cytogenetic Nomenclature (ISCN 2013).

Below is a brief description of the chromosome groups and morphology. These characteristics guide the chromosomal analysis from the methodology used.

The chromosomes are divided into 7 groups, A, B, C, D, E, F, and G, and are classified according to their size and the position of the centromere. The centromere is located at the center of the Metacentric chromosomes, slightly above the center in the Submetacentric chromosomes and almost in the extremities in the Acrocentric chromosomes.
Group A: are the largest chromosomes, being pairs 1, 2 and 3. The classification of the chromosomes in this group is: 1 and 3 metacentric pairs and 2 submetacentric pairs.
Group B: are the major submetacentric chromosomes, being pairs 4 and 5.
Group C: are the submetacentric chromosomes, being pairs 6, 7, 8, 9, 10, 11, 12 and X.
Group D: are the largest acrocentric chromosomes, being pairs 13, 14 and 15.
Group E: are the smallest submetacentric chromosomes, being pairs 16, 17 and 18.
Group F: are the smallest metacentric chromosomes, being pairs 19 and 20.
Group G: are the smallest submetacentric chromosomes, being the pairs 21, 22 and Y.

A standard karyotype with some practical tips on how to identify chromosomes during an analysis is attached, but no information contained in this annex should replace the international chromosome standards described in: International System For Human Cytogenetic Nomenclature (ISCN 2013).

Preparation for Verification of the Genetic Control

The preparation for carrying out the verification of genetic control consists in checking the equipment, software and materials required for this purpose, as described below:

Materials Used:
Genetic control medium (GCM);
Synchronization Solution (Vinblastine);
Cellular Deployment Solution;
Hypotonic Solution (KCL 0.075M);
Trypsin 0.025%;
Phosphate Buffer Solution 0.06M pH 6.8 (Potassium Phosphate Monobasic and Sodium Phosphate Dihydrate);
Coloring Solution with Phosphate Buffer 0.03M pH 6.8 (Potassium Monobasic Phosphate and Sodium Phosphate Dibasic Dihydrate);
Fixing solution (3:1 methanol and acetic acid).

Inanimate Material:
25 cm2 culture bottle;
15 mL sterile falcon type tube;
Sterile 1.5 mL microtube with lid;
Sterile 3 mL Pasteur pipette;
Glass Pasteur Pipette;
10 mL Serological Pipette;
Slide streaked and frosted for microscopy;
Lamp.

The slides for microscopy should have a streaked and frosted end, properly washed according to the procedure below:

Slide Cleaning:
With a gauze slightly soaked in 70% Alcohol, clean the entire surface of each slide.
Then pass a dry gauze over the entire surface of the slide until it is dry and without stains.
Slides with small surface defects should be discarded in order to avoid compromising the analysis.

Inoculation of Cells with Culture Medium:
At the time of cryopreservation, remove two 0.5 mL aliquots from the tube identified as Q.C. and distribute to two 25 cm bottles identified with labels containing the beneficiary ID, the current sample passage and the Karyotype I (Replicate I) identification for one of the bottles and Karyotype II (Replicate II) for the other, thus maintaining the traceability of the bottle that will be sent to the responsible in Q.C. (ISO8).
If the cells are frozen, proceed according to the Biological Sample Defrost procedure.
After thawing, inoculate the cells in 2 mL of Genetic Control Medium (GCM) in a 25 cm² bottle and place in an incubator at 37° C. and 5% CO2 for growth.
In the cases where the cells were thawed, between 3-10 days of growth, with confluence of approximately 80 to 90%, make a Split in a 25 cm² bottle.
At this point, proceed with only one bottle of culture, preserving the second for possible repetition.
Between 1 and 3 days of growth, with confluence of approximately 45 to 60%, add 0.04 mL of the Synchronization Solution to the culture medium and incubate for 6 hours at 37° C. and 5% CO2.
Highlight the cells using 1.5 mL of Cellular Detachment.
Inactivate the enzyme of detachment with 3 mL of basal medium.
Transfer the cells to a 15 mL falcon tube, identified with a pre-printed label, containing the beneficiary ID, withdrawal date, identification of which of the bottles will be sent to Q.C. (Karyotype I or II) and the current bottle number.
Transfer the tube to the person in Q.C. (ISO8), who will finish the procedure.
Centrifuge for 5 min at 1500 rpm or 220G.
Remove the supernatant with the aid of a Pasteur pipette.
Add 5 mL of Hypotonic Solution (KCL 0.075M) previously heated to 37° C., resuspend the cells and place in a greenhouse or water bath at 37° C. for 15 to 30 minutes.
Add 1 mL of Fixing Solution to stop the action of the Hypotonic Solution.
Centrifuge for 5 minutes at 1500 rpm or 220G.
Discard the supernatant and put 5 mL of fixing.
Centrifuge for 5 minutes at 1500 rpm or 220G.
Discard the supernatant and resuspend the material in approximately 2 mL of Fixing Solution (depending on the amount of pellet obtained) for further analysis.

Preparation of the Slides:
Two slides, identified by a black pencil with the beneficiary 1D, the slide number and the withdrawal date, must be prepared for each sample.
From this point, proceed with the appropriate PPE (safety glasses and activated carbon semi facial mask).
With the lamp already on, drip between 4 and 6 drops (approximately 0.4 mL, which may vary according to the amount of pellet obtained) of the material on a lean and identified slide.
Pass the slide quickly in the heat of the flame of the lamp (without getting to close to the flame) and place it on a flat surface to dry at room temperature.
At this point, the slides can be stored in an incubator at 37° C. until the next work day, or continue with the procedure for checking the index of metaphases in the sequence, just waiting for the slides to be COMPLETELY dry.
Perform conventional staining with Coloring Solution (0.03M Phosphate Buffer and 5% Methylene Blue dye).
Observe the obtained metaphase index under microscope.
Obtaining a number greater of 4 metaphases with a good chromosome stretch pattern (which can be analysed) in the two prepared slides, perform the procedure to prepare another 3 slides. Otherwise, continue with the culture in the Processing (ISO7).
Leave the slides for 24 hours in an incubator at 37° C. or at room temperature for 3 days, so that the chromosomes are fixed.
After this period, store the slides in a suitable container and identified in a refrigerator at a temperature between 2 and 8° C.
Complete the volume of the tube containing the remaining material with 2 mL of Fixing Solution and store it in a freezer at between −5 to −40° C. until the sample is analysed and report has been issued.
After the report is issued, discard the slides and the tube with the extra sample.

GTG Banding Technique

To obtain G-band, the modified technique of Seabright (1971) will be used.
Soak the slide to be analysed in a Banding Solution (0.025% Trypsin) previously heated in a water bath at 37 C for two to three seconds.
Rinse the slide with potable water.
In a suitable container, stain the slide with Coloring Solution (0.03M Phosphate Buffer and 5% Methylene Blue dye) for 3 minutes.
Rinse the slide with potable water.

Karyotype Analysis

To do this, proceed as described below:
Turn on the microscope.
Adjust the light intensity.
Focus on metaphase with macrometric and micrometric (lens of 10× and 40×).
Set the 100× lens and immersion oil.
Analyse 10 metaphases and to document in the Analysis of Metaphase Record, the documentation can be done manually or printed.

Chromosomal Analysis

The Geneall automatic cytogenetic analysis system will be used to process the karyotype image. Ten metaphases will be analysed in microscopy, accompanied by the record of metaphase analysis. Of these 10, two will be photographed and the karyotype set. To do this, proceed as described below.

Image record when photographing a metaphase, follow the steps below:

Procedure:
Within the patient folder, create a folder identified as Quality Control.
Within the Quality Control folder, create a folder labelled “GENETIC CONTROL”.
In this folder, identify the photo in the File Name field as the example: ID-n. of metaphase. In the Type field, select the bmp format and click Save.
To analyse the chromosomes it is necessary to use a software to separate the chromosomes.
Open a photo of the metaphase being processed and cut out the chromosomes.
Group the chromosomes according to the international standard (ISCN 2013) and assemble the karyotype.
If the sample already has the mentioned folders, proceed normally and save the sample.

The program will create an automatic file in the folder.

The Records will be presented as follows:
Metaphase Analysis Record Form:

Genetic Control Historic:

Genetic Control Report:

Thawing

The cryopreserved cell thawing procedure is performed if samples are requested for patient referral or other quality control analysis.

PROCEDURE

Preparation for Sample Thawing:

Inanimate Materials:
03 sterile graduated pipettes;
03 sterile Pasteur pipettes;
01 package of sterile wiper;
01 15 mL falcon tube,
01 50 mL sterile falcon tube;
01 25 cm² culture bottle;

Solutions:
2 mL of Complete Xeno-free Medium or GCM Medium;
5 mL of Basal Medium;
2 mL of Solution B.

Equipment Verification

All equipment that will be part of the process shall be checked prior to sample processing for proper operation, cleaning and general maintenance. Below are listed the equipment that will be part of the process.
Biological safety cabinet;
Water bath;
Centrifuge;
CO2 incubator;
Semi-automatic pipette;
Stopwatch.

Sample Thawing

After completing the preparation step, ask the collaborator responsible for the cryopreservation sector for the sample to be thawed and proceed as described below.
Check the integrity of the packaging and record the information on the Cell Thawing Record Form.

Cell Thawing Record Form:
Place the cryotube in the Water Bath at 37° C.
With circular movements, leave the tube in the bath for 3 minutes or until 80-90% of the solution has been liquefied leaving only traces of the frozen solution.
Take the tube immediately to the Biological Safety Cabinet.
Clean the cryotube with sterile wiper.
Using a pipette, transfer the volume of the cryotube to the tube containing 5 mL of Basal medium.
Homogenize and centrifuge for 5 minutes at 1000 RPM or 178G.
Remove the tube, take to the cabinet and dispense the supernatant.
Resuspend the pellet with 2 mL of Solution B.
Homogenize and centrifuge for 5 minutes at 1000 RPM or 178 G.
Remove the tube, take to the cabinet and dispense the supernatant.
Resuspend the pellet with 2 mL of Complete Xeno-free Medium or GCM Medium, the latter being used for samples that will be submitted to a Quality Control test.
Transfer to 25 cm2 bottle and incubate at 37° C. and 5% CO2 atmosphere.
Proceed according to the Item Tracking and Medium Change.

Traceability of Information

Thawing Is recorded In the Biological Sample Thawing Record.

Criteria for Acceptance of Processed Samples

This item establishes the acceptance and rejection criteria for samples during all stages of the process.

Procedure

During all stages of sample processing, there are specific criteria which we call acceptance criteria for the sample, so that it is approved to follow the steps thus guaranteeing the quality of the process.

If the sample does not meet one of the criteria, regardless of the process step, the sample is invalidated and can no longer be processed and must be discarded.

Only samples that meet all acceptance criteria will be cryopreserved.

Sample processing steps involving acceptance criteria are listed below:
Receipt of the Sample;
Monitoring and Medium Change;
Cell adhesion;
Immunophenotyping;
Karyotyping;
Cell Differentiation;
Sterility.

Below are the established criteria for each stage.

Acceptance Criteria for Each Step:

| Processing Stage | Acceptance criteria | Stage characteristics |
|---|---|---|
| Receipt of the | Complete identification of | In this step the transport parameter and the intrinsic |
| Sample | the sample | parameters of the sample are verified |
| Monitoring and | Absence of sample | This step occurs throughout the expansion process, in |
| Medium Change | contamination | which the sample is checked periodically |
| Cell Adhesion | Cell adhesion in up to 24 | Cellular adhesion is a critical step during the cell culture |
| | days after start of | process, since only after cell adhesion in the bottle they |
| | incubation | begin the multiplication step |
| Immunophenotyping | CD 105: >90% | Immunophenotyping is an assay performed to certify that the |
| | CD 73: >90% | cell surface receptors, characteristic of the stem cell of the |
| | CD 146: >60% | pulp of baby tooth, remained intact during the process of cell |
| | CD 45: <10% | expansion |
| Karyotyping | 46 XX for women | Karyotyping is a genetic test performed to check if there was |
| | 46 XY men | any chromosomal alteration in the cells during the expansion |
| | | process. |
| Cell Differentiation | Confirmed ability to | The cell differentiation test is performed to verify the ability |
| | differentiate into | of baby stem cells from the pulp of baby tooth to differentiate |
| | bone tissue | into bone tissue |
| Sterility | Sterile | The sterility test is performed to verify the presence of |
| | | contamination in the sample, that is, it certifies that the |
| | | cryopreserved sample has no microorganisms. |

Availability of Cells for Research or Therapy

Establish a standard for the release of stored cells for research or therapy.

### Definitions and Abbreviations

Ethics and Research Committees (CEP): Interdisciplinary and independent colleges, with public munus, of an advisory, deliberative and educational character, created to defend the research subjects' interests and their integrity and dignity and to contribute to the development of research within ethical standards.

National Commission for Research Ethics (CONEP/MS): a collegiate and independent body of an advisory, deliberative, normative and educational nature, linked to the National Health Council.

NIH: National Institute of Health; FDA—Food and Drug Administration.

EMA—European Medicine Agency.

Procedure

Human cells and their derivatives may only be made available upon proof of approval for the clinical research by the CEP/CONEP system or proof that the therapeutic procedure is authorized by the Federal Council of Medicine (CFM) or Federal Council of Dentistry (CFO). In each country where there is a similar operation initiative, local legislation should be followed.

After this verification and if there is the need to use the cells of a beneficiary for both research and therapy, the collaborator in the laboratory area may send these cells in two ways, as described in the items below:

Procedure for Sample Shipping with Thawing

Upon request of cells already in the expansion phase, the laboratory will proceed as described in the Thawing of Biological Samples and recorded on the Biological Samples Thawing Record Form.

The samples will be expanded according to processes already carried out in the company.

The amount of cells and the way they will be delivered (quantity, undifferentiated, partially differentiated, fully differentiated, filtered into specific subpopulations) should be in accordance to the Center for Technology. Therapy or Cellular Research to which cells will be send and to what is described in the project CEP/CONEP or according to the local legislation depending on the country to where the cells will be delivered. In order to carry out this procedure in another country, the legislation and way of delivery must follow the one recommended by the current norm.

Procedure of Sipping Frozen Sample

At the request of the frozen cells to be sent to the Cell Technology Center, it must be qualified by the quality control department and the R-Crio's Technical Manager or its substitute.

The sample will be sent through the Voyager cylinder provided by R-Crio. The thawing of this sample shall be performed according to R-Crio.

The CTC qualification will be reported in the CTC Qualification Report.

1. PROCESS OF CRYOPRESERVATION OF STEM CELLS OBTAINED FROM THE PULP OF DECIDUOUS TEETH comprising:
COLLECTION OF STEM CELLS FROM DECIDUOUS TEETH during the extraction process, and being constituted of three phases, namely collection (phase 1), shipping (phase 2) and processing (phase 3): QUALITY CONTROL, consisting of five quantitative and qualitative analysis tests, namely: 1) CELL COUNT AND VIABILITY, 2) STERILITY, 3) INDUCTION OF MULTIPOTENCY WITH OSTEOGENIC DIFFERENTIATION, 4) KARYOTYPING AND 5) IMMUNOPHENOTYPING BY FLOW CYTOMETRY: CRYOPRESERVATION: THAWING AND SHIPPING OF THE STEM CELLS to therapy and/or research centers in accordance with current local and international legislation and with the specificities related to the demand for use of stem cells.
Phase 1 (Collection):
It comprises all the steps described herein, preparatory and corresponding to the collection of the deciduous tooth. The requested tests, the professional competence, the appropriate conditions for the extraction of the dental element aiming at a minimally invasive procedure, the technique of collection in which the risk of contamination is prevented and the guarantee of traceability of the sample, the formatted R-Crio's questionnaire and packaging of the sample.
Phase 2 (Shipping):
Described in the text: the sample preparation according to a validated methodology, in which after the extraction de dental element is conditioned in a specific medium to maintain cell viability, indelible and fully traceable identification material, special and validated thermal box, keeping its conditions for twice the time recommended in the process.
Phase 3 (Processing):
Comprises the receipt of the deciduous tooth in accordance with the previous steps of collection, packaging and shipping, all steps of preparation of supplies, records, preparation of the tooth for removal and digestion of the pulp in order to allow the isolation of the mesenchymal stem cells and subsequent expansion of these cells by systematizing the information and processing of the sample at an appropriate period. Record model that allows the processing of the sample considering a time interval of up to 48 hours between the collection of the tooth and its arrival at the Center of Cellular Technology for processing. Method that guarantees asepsis and isolation of cells. Preparation of specific materials and detail of the process. Methods of traceability of deviations in the process.

2. PROCESS OF CRYOPRESERVATION OF STEM CELLS OBTAINED FROM THE PULP OF DECIDUOUS TEETH corresponding to the step of QUALITY CONTROL. Methodologies described to guarantee quantitatively and qualitatively the viability of the stem cells that will be cryopreserved for later application in cellular therapies or research. QUALITY CONTROL, consisting of five quantitative and qualitative analysis tests, namely: 1) CELL COUNT AND VIABILITY, 2) STERILITY, 3) INDUCTION OF MULTIPOTENCY WITH OSTEOGENIC DIFFERENTIATION AND 5) IMMUNOPHENOTYPING BY FLOW CYTOMETRY. Aims, methodologies, specific preparation of solutions and reagents and sets of unique techniques to guarantee the complete preservation of cell characteristics.

3. PROCESS OF CRYOPRESERVATION OF STEM CELLS OBTAINED FROM THE PULP OF DECIDUOUS TEETH corresponding to the step of CRYOPRESERVATION. Cryopreservation involves the formulation of an exclusive medium for the maintenance of the cells over time. It involves the optimal concentration of components, cells, and techniques that ensure cell viability. The temperature decay ramp maintains the high viability of the cells after thawing.

4. PROCESS OF CRYOPRESERVATION OF STEM CELLS OBTAINED FROM THE PULP OF DECIDUOUS TEETH corresponding to the steps of THAWING AND SHIPPING.
This process defines the techniques of thawing a sample in the process. This technique allows the cells to be thawed after undefined storage periods at low temperature maintaining their high viability. The delivery is made after the process of thawing and expansion of the cells while still maintaining their characteristics and cellular plasticity and must be in accordance with the local laws in force.
